(19) United States
(12) Patent Application Publication
(10) Pub. No.: US 2022/0326258 A1
(43) Pub. Date: Oct. 13, 2022

(54) BIOMARKERS FOR AMYOTROPHIC LATERAL SCLEROSIS (ALS) AND MOTOR NEURON DISEASES

(71) Applicant: Northwestern University, Evanston, IL (US)

(72) Inventor: Pembe Hande Ozdinler, Chicago, IL (US)

(21) Appl. No.: 17/642,374

(22) PCT Filed: Sep. 11, 2020
(86) PCT No.: PCT/US2020/050415

§ 371 Date: Mar. 11, 2022

Related U.S. Application Data
(60) Provisional application No. 62/899,854, filed on Sep. 13, 2019.

(51) Int. Cl. G01N 33/68

(52) U.S. Cl. CPC G01N 33/6896; G01N 2800/52

(57) ABSTRACT

Provided herein is a method comprising detecting and quantifying the amount of at least two proteins in a serum sample obtained from a subject suffering from a motor neuron disease, such as amyotrophic lateral sclerosis (ALS). The method may be used to treat the subject by measuring a change in concentration of the at least two or more proteins in the serum sample over a duration of time and administering a treatment to the subject based upon the change in concentration. The two or more proteins may be APOC3, APOF, C8B, C8G, IGHG3, ITIH3, QSOX1, SERPINA10, SERPINA5, VWF, APOA2, APOA4, APOD, APOL1, C4B, CLEC3B3, CLU, APCS, BCHE, CIR, CFH, GP1BA, PROS1, SERPINA4, SEPP1, A2M, AGT, C1RL, CD14, FCN2, SERPINA1, SERPINF2, APOA1, and/or IGFBP3.

### CROSS-REFERENCE TO RELATED APPLICATIONS

This application claims the benefit of U.S. Provisional Patent Application No. 62/899,854, filed on Sep. 13, 2019, which is incorporated by reference herein.

### FIELD

The disclosure is directed to methods for detecting and quantifying the amount of at least two proteins in a serum sample obtained from a subject suffering from a motor neuron disease, such as amyotrophic lateral sclerosis (ALS). The methods described herein may be used, for example, to monitor the progression of a motor neuron disease, to select a treatment regimen for a motor neuron disease, and to monitor drug responsiveness of a patient suffering from a motor neuron disease.

### BACKGROUND OF THE INVENTION

Amyotrophic lateral sclerosis (ALS), also known as Lou Gehrig's disease, is a fatal neurodegenerative disease characterized by the loss of upper and lower motor neurons. Affected individuals develop progressive muscle weakness and atrophy, eventually leading to death due to respiratory failure (Sreedharan J. and Brown R H., Jr., Ann. Neurol., 74: 309-316 (2013); and Kiernan et al., Lancet, 377: 942-955 (2011). ALS may be classified as a motor neuron disease (MND). Motor neuron diseases (MNDs) are a group of progressive neurological disorders that destroy motor neurons, affecting speech, movement, breathing, and swallowing.

Decades of basic research and clinical studies have provided initial insight into the pathogenic mechanisms of selective motor neuron degeneration; however, currently there are no specific diagnostic tests for most MNDs, including biomarkers, and no effective cures or long-term solutions for MDNs, especially ALS.

There remains a need for methods for treating motor neuron diseases such as ALS, as well as methods for monitoring progression of MNDs and assessing responsiveness of patients to potential treatments for MNDs.

### BRIEF SUMMARY OF THE INVENTION

Provided herein is a method comprising detecting and quantifying the amount of at least two proteins in a serum sample obtained from a subject suffering from a motor neuron disease, which two or more proteins are selected from apolipoprotein C3 (APOC3), apolipoprotein F (APOF), complement C8 beta chain (C8B), complement C8 gamma chain (C8G), immunoglobulin heavy constant gamma 3 (IGHG3), inter-alpha-trypsin inhibitor heavy chain 3 (ITIH3), quiescin sulfhydryl oxidase 1 (QSOX1), SERPINA10, SERPINA5, von Willebrand factor (VWF), apolipoprotein A2 (APOA2), apolipoprotein A4 (APOA4), apolipoprotein D (APOD), apolipoprotein L1 (APOL1), complement C4-B (C4B), C-type lectin domain family 3 member B (CLEC3B), clusterin (CLU), amyloid P component, serum (APCS), butyrylcholinesterase (BCHE), complement C1R (CIR), complement factor H (CFH), glycoprotein 1b platelet subunit alpha (GP1BA), protein S (PROS1), SERPINA4, selenoprotein P (SEPP1), alpha-2-Macroglobulin (A2M), angiotensinogen (AGT), complement C1r subcomponent like (C1RL), cluster of differentiation 14 (CD14), ficolin-2 (FCN2), SERPINA1, SERPINF2, apolipoprotein A1 (APOA1), and insulin like growth factor binding protein 3 (IGFBP3).

Also provided herein is a method of treating a subject suffering from a motor neuron disease. The method comprises (a) detecting and quantifying the amount of at least two proteins in a serum sample obtained from a subject suffering from a motor neuron disease, which two or more proteins are selected from apolipoprotein C3 (APOC3), apolipoprotein F (APOF), complement C8 beta chain (C8B), complement C8 gamma chain (C8G), immunoglobulin heavy constant gamma 3 (IGHG3), inter-alpha-trypsin inhibitor heavy chain 3 (ITIH3), quiescin sulfhydryl oxidase 1 (QSOX1), SERPINA10, SERPINA5, von Willebrand factor (VWF), apolipoprotein A2 (APOA2), apolipoprotein A4 (APOA4), apolipoprotein D (APOD), apolipoprotein L1 (APOL1), complement C4-B (C4B), C-type lectin domain family 3 member B (CLEC3B), clusterin (CLU), amyloid P component, serum (APCS), butyryicholinesterase (BCHE), complement C1R (C1R), complement factor H (CFH), glycoprotein 1b platelet subunit alpha (GP1BA), protein S (PROS1), SERPINA4, selenoprotein P (SEPP1), alpha-2-Macroglobulin (A2M), angiotensinogen (AGT), complement C1r subcomponent like (C1RL), cluster of differentiation 14 (CD14), ficolin-2 (FCN2), SERPINA1, SERPINF2, apolipoprotein A1 (APOA1), and insulin like growth factor binding protein 3 (IGFBP3), (b) repeating step (a) at specific time points over a duration of time and measuring a change in concentration of the at least two or more proteins in the serum sample over the duration of time, and (c) administering a treatment to the subject based upon the change in concentration of the at least two or more proteins measured in step (b).

The disclosure also provides method of treating a subject suffering from a motor neuron disease, which method comprises: (a) detecting and quantifying the amount of at least two proteins in a serum sample obtained from a subject suffering from a motor neuron disease, which two or more proteins are selected from apolipoprotein C3 (APOC3), apolipoprotein F (APOF), complement C8 beta chain (C8B), complement C8 gamma chain (C8G), immunoglobulin heavy constant gamma 3 (IGHG3), inter-alpha-trypsin inhibitor heavy chain 3 (ITIH3), quiescin sulfhydryl oxidase 1 (QSOX1), SERPINA10, SERPINA5, von Willebrand factor (VWF), apolipoprotein A2 (APOA2), apolipoprotein A4 (APOA4), apolipoprotein D (APOD), apolipoprotein L1 (APOL1), complement C4-B (C4B), C-type lectin domain family 3 member B (CLEC3B), clusterin (CLU), amyloid P component, serum (APCS), butyrylcholinesterase (BCHE), complement C1R (C1R), complement factor H (CFH), glycoprotein 1b platelet subunit alpha (GP1BA), protein S (PROS1), SERPINA4, selenoprotein P (SEPP1), alpha-2-Macroglobulin (A2M), angiotensinogen (AGT), complement C1r subcomponent like (C1RL), cluster of differentiation 14 (CD14), ficolin-2 (FCN2), SERPINA1, SERPINF2, apolipoprotein A1 (APOA1), and insulin like growth factor binding protein 3 (IGFBP3), (b) detecting an increase or decrease of each of the two or more proteins in the serum sample as compared to the amounts of the same two or more proteins in a control sample, and diagnosing the subject as having a motor neuron disease; and (c) administering to the subject a treatment for the motor neuron disease.

### BRIEF DESCRIPTION OF THE DRAWING(S)

FIG. 1A is a graph showing increased SEPP1 protein levels in the serum of ALS patients with prominent upper motor neuron loss as compared to healthy controls. FIG. 1B is a graph showing SEPP1 protein levels detected in each female ALS patient with respect to female controls. FIG. 1C is a graph showing SEPP1 protein levels detected in each male ALS patient with respect to male controls. In FIGS. 1B and 1C, the y-axis shows the log 2-transformed fold-change between patient and control. The x-axis shows the patient.

FIG. 2A is a graph showing increased SERPINA5 protein levels in the serum of ALS patients with prominent upper motor neuron loss as compared to healthy controls. FIG. 2B is a graph showing SERPINA5 protein levels detected in each female ALS patient with respect to female controls. FIG. 2C is a graph showing SERPINA5 protein levels detected in each male ALS patient with respect to male controls. In FIGS. 2B and 2C, the y-axis shows the log 2-transformed fold-change between patient and control. The x-axis shows the patient.

FIG. 3A is a graph showing APOA4 protein levels detected in each female ALS patient with respect to female controls. FIG. 3B is a graph showing APOA4 protein levels detected in each male ALS patient with respect to male controls. The y-axis shows the log 2-transformed fold-change between patient and control. The x-axis shows the patient.

FIG. 4A is a graph showing APOF protein levels detected in each female ALS patient with respect to female controls. FIG. 4B is a graph showing APOF protein levels detected in each male ALS patient with respect to male controls. The y-axis shows the log 2-transformed fold-change between patient and control. The x-axis shows the patient.

FIG. 5A is a graph showing CLU protein levels detected in each female ALS patient with respect to female controls. FIG. 5B is a graph showing CLU protein levels detected in each male ALS patient with respect to male controls. The y-axis shows the log 2-transformed fold-change between patient and control. The x-axis shows the patient.

FIG. 6A is a graph showing C1R protein levels detected in each female ALS patient with respect to female controls. FIG. 6B is a graph showing C1R protein levels detected in each male ALS patient with respect to male controls. The y-axis shows the log 2-transformed fold-change between patient and control. The x-axis shows the patient.

FIG. 7A is a graph showing PROS1 protein levels detected in each female ALS patient with respect to female controls. FIG. 7B is a graph showing PROS1 protein levels detected in each male ALS patient with respect to male controls. The y-axis shows the log 2-transformed fold-change between patient and control. The x-axis shows the patient.

FIG. 8A is a graph showing SERPINF2 protein levels detected in each female ALS patient with respect to female controls. FIG. 8B is a graph showing SERPINF2 protein levels detected in each male ALS patient with respect to male controls. The y-axis shows the log 2-transformed fold-change between patient and control. The x-axis shows the patient.

### DETAILED DESCRIPTION OF THE INVENTION

The present disclosure is predicated, at least in part, on the discovery of protein biomarkers that indicate the progression of motor neuron diseases, such as ALS, and the extent of motor neuron loss in affected patients. In this regard, provided herein are methods comprising detecting and quantifying the amount of at least two proteins in a serum sample obtained from a subject suffering from a motor neuron disease treating of treating a subject suffering from a motor neuron disease, which methods can be performed over a duration of time to determine a change in concentration of the at least two proteins. Such methods can be used to select an appropriate treatment for the subject, to monitor the progression of disease in the subject, and/or to monitor a subject's response to a particular treatment.

For example, the disclosure describes the identification of two proteins, SEPP1 and SERPINA5, that broadly inform whether upper motor neurons (UMNs) are involved in a motor neuron disease. Proteins were also identified that inform on the underlying causes of UMN vulnerability, such as lipid homeostasis defects (e.g., APOA4, APOF), neuroimmune modulations (e.g., C1R, CFH, PROS1), defects in cytoarchectural dynamics and integrity (e.g., SERPINF2), oxidative stress, and endoplasmic reticulum (ER) stress (e.g., CLU, SEPP1). The identification of these protein biomarkers and the change in their levels over time may guide the development of novel and patient-based treatment strategies for ALS and other motor neuron diseases

### Definitions

The terms “biomarker” and “biological marker” are used synonymously herein and refer to a defined characteristic that is measured as an indicator of normal biological processes, pathogenic processes, or responses to an exposure or intervention, including therapeutic interventions. In some embodiments, a biomarker may comprise a substance whose detection indicates a particular disease state (e.g., the presence of an antibody may indicate an infection). More specifically, a biomarker may indicate a change in expression or state of a protein that correlates with the risk or progression of a disease, or with the susceptibility of the disease to a particular treatment. The types of substances that may be measured as biomarkers range widely and include, but are not limited to, molecular biomarkers (e.g., nucleic acids, gene products, and proteins), physiologic biomarkers (e.g., blood pressure or blood flow), or anatomic biomarkers (e.g., the structure of a particular organ).

The terms “increased,” “increase,” and “elevated” may be used interchangeably herein and refer to an amount or a concentration in a sample that is higher or greater than a predetermined level or range, such as a typical or normal level found in a control group or control sample, or is higher or greater than another reference level or range (e.g., earlier or baseline sample). The terms “decreased,” “decrease,” “lowered,” and “reduced” may be used interchangeably herein and refer to an amount or a concentration in a test sample that is lower or less than a predetermined level or range, such as a typical or normal level found in a control group or control sample, or is lower or less than another reference level or range (e.g., earlier or baseline sample). The term “altered” refers to an amount or a concentration in a sample that is altered (increased or decreased) over a predetermined level or range, such as a typical or normal level found in a control group or control sample, or over another reference level or range (e.g., earlier or baseline sample).

As used herein, “motor neuron diseases (MNDs)” refers to a clinically and pathologically heterogeneous group of neurologic diseases characterized by progressive degeneration of motor neurons, which includes both sporadic and hereditary diseases. MNDs may involve either or both of upper motor neurons (UMNs) and lower motor neurons (LMNs). Upper motor neurons originate from the primary motor cortex of the cerebrum (precentral gyrus) and possess long axons forming corticospinal and corticobulbar tracts, while lower motor neurons (LMNs), which originate in the brainstem (cranial nerve [CN] motor nuclei) and spinal cord (anterior horn cells) and directly innervate skeletal muscles. Upper motor neurons direct the lower motor neurons to produce movements such as walking or chewing. Lower motor neurons control movement in the arms, legs, chest, face, throat, and tongue. Upper motor neuron degeneration is a hallmark of many motor neuron diseases in which voluntary movement is impaired.

MNDs typically are classified according to whether they are inherited or sporadic, and to whether degeneration affects upper motor neurons, lower motor neurons, or both. In adults, the most common MND is amyotrophic lateral sclerosis (ALS), which affects both upper and lower motor neurons. Other types of MNDs include, but are not limited to progressive bulbar palsy, pseudobulbar palsy, primary lateral sclerosis (PLS), progressive muscular atrophy, spinal muscular atrophy (SMA), hereditary spastic paraplegia (HSP), post-polio syndrome (PPS), or X-linked spinobulbar muscular atrophy (Kennedy disease).

ALS, also known as Lou Gehrig disease, is a fatal disorder characterized by progressive skeletal muscle weakness and wasting or atrophy (i.e., amyotrophy), spasticity, and fasciculations as a result of degeneration of the UMNs and LMNs, culminating in respiratory paralysis. In general, ALS is classified as sporadic, familial, or Western Pacific ALS with or without Parkinsonism-dementia complex (ALS/PDC). Most ALS cases are sporadic, and only 5-10% of cases are considered to be familial. Mutations in the C9orf72 gene are responsible for 30-40% of familial ALS cases in the United States and Europe. Worldwide, approximately 20% of cases of familial ALS are due to a mutation in the Cu/Zn superoxide dismutase-1 gene (SOD1). Western Pacific ALS occurs on the islands of Guam (Guam ALS), on the Kii peninsula of Japan, and in Western New Guinea. It is now clear that a subset of ALS cases shows features of frontotemporal lobar degeneration (FTLD) (i.e., FTLD-MND/ALS). Another category of ALS that predominantly involves upper motor neuron symptoms has been recognized and referred to as “upper motor neuron-dominant” ALS (UMN-dominant ALS). UMN-dominant ALS may be defined as ALS due predominantly to UMN signs but with minor electromyogram (EMG) denervation or LMN signs on examination (Soraru et al., Amyotroph Lateral Scler., 11(5): 424-9 (2010)).

Progressive bulbar palsy (PBP) is a progressive degenerative disorder of the motor nuclei in the medulla (specifically involving the glossopharyngeal, vagus, and hypoglossal nerves) that produces atrophy and fasciculations of the lingual muscles, dysarthria, and dysphagia. In adults, because most of the cases presenting with these pure bulbar symptoms represent so-called bulbar-onset ALS and eventually develop widespread symptoms typically seen in ALS, some authors consider this disorder to be a subset of ALS. Infantile PBP is a rare disorder that occurs in children and presents as the following 2 phenotypically associated forms: Brown-Vialetto-Van Laere syndrome (pontobulbar palsy with deafness) and Fazio-Londe disease. Brown-Vialetto-Van Laere syndrome is characterized by bilateral sensorineural deafness that is followed by CN VII, CN IX, and CN XII palsies, whereas Fazio-Londe disease causes progressive bulbar palsy without deafness. Both disorders are genetically heterogeneous.

Pseudobulbar palsy is a condition typically caused by bilateral damage to corticobulbar pathways, which are upper motor neuron pathways that course from the cerebral cortex to nuclei of cranial nerves in the brain stem. Pseudobulbar palsy patients experience difficulty chewing and swallowing, have increased reflexes and spasticity in tongue and the bulbar region, and demonstrate slurred speech (which is often the initial presentation of the disorder). Some patients also experience uncontrolled emotional outbursts.

Primary lateral sclerosis (PLS) is a rare, idiopathic neurodegenerative disorder that primarily involves the UMNs, resulting in progressive spinobulbar spasticity. Because substantial numbers of cases initially diagnosed as PLS would be reclassified as ALS as the disease progresses, Pringle et al suggest that a disease duration of at least 3 years is required to render this diagnosis clinically (Pringle et al., Brain, 115 (Pt 2): 495-520 (1992)). There is still debate regarding whether PLS is a distinct pathologic entity or whether it represents one end of a clinical spectrum of ALS.

Progressive muscular atrophy (PMA), also known as Duchenne-Aran muscular atrophy, is a rare, sporadic, adult-onset motor neuron disease, clinically characterized by isolated lower motor neuron features; however, clinically evident upper motor neuron signs may emerge in some patients. PMA is regarded as a heterogeneous syndrome showing considerable overlap with ALS (Vissner et al., Arch Neurol. 2007; 64(4): 522-528). As a result of lower motor neuron degeneration, the symptoms of PMA include, for example, atrophy, fasciculations, and muscle weakness. Some patients have symptoms restricted only to the arms or legs (or in some cases just one of either). These cases are referred to as “Flail Arm” (FA) or “Flail Leg” (FL) and are associated with a better prognosis (Wijesekera et al., Neurology, 72(12): 1087-1094 (2009)).

Spinal muscular atrophy (SMA) comprises a large group of genetically determined neuromuscular disorders that are characterized by progressive degeneration of spinal LMNs (i.e., alpha motor neurons in the anterior horns) accompanied by amyotrophy, with no evidence of sensory or pyramidal tract involvement. This condition is genetically heterogeneous, with autosomal recessive (most common), autosomal dominant, and X-linked recessive modes of inheritance. The International SMA Consortium defined the following 4 clinical groups, depending on the age of onset and achieved motor abilities (Munsat T. L. and Davies K. E., Neuromuscul Disord., 2(5-6): 423-8 (1992)): Type I SMA (acute form, Werdnig-Hoffmann disease), Type II SMA (intermediate form), Type III SMA (juvenile form, Kugelberg-Welander disease), and Type IV SMA (adult form).

Hereditary spastic paraparesis (HSP), also known as familial spastic paraplegias or Strumpell-Lorrain disease, comprises a clinically and genetically heterogeneous group of hereditary disorders characterized by slowly progressive spastic paraparesis. This condition is clinically classified as taking either a pure (uncomplicated) form or a complicated form, depending on whether the paraparesis exists in isolation or in conjunction with other major clinical features. There is significant overlap in clinical characteristics between pure HSP and PLS (Brugman et al., Arch Neurol., 66(4): 509-14 (2009)). Genetically, HSPs are classified by the mode of inheritance (autosomal dominant, autosomal recessive, and X-linked) and are subdivided by chromosomal locus or causative gene. Pure autosomal dominant HSP is the most common form.

Post polio syndrome (PPS) is a clinical diagnosis and essentially one of exclusion. This condition is characterized by late-onset muscle weakness and fatigue in skeletal or bulbar muscles, unrelated to any known cause, in individuals with a previous history of an acute attack of paralytic poliomyelitis (Dalakas M C, Ann NY Acad Sci. 1995: 753:68-80). Survivors of the polio epidemics that occurred worldwide in the mid-20th century constitute a population at risk for PPS.

First described in Kennedy et al., Neurology, 18(7): 671-80 (1968), X-linked spinobulbar muscular atrophy (SBMA), also known as Kennedy disease, is an adult-onset, X-linked recessive trinucleotide, polyglutamine disorder that is caused by expansion of a polymorphic CAG tandem-repeat in exon 1 of the androgen-receptor gene on chromosome Xq11-12 (Finsterer, J., Eur. J. Neurol., 16(5): 556-61 (2009)). This disorder is characterized by slowly progressive weakness of bulbar and limb muscles, associated with endocrinologic disturbances (androgen insensitivity). Because of X-linked transmission, this disorder almost exclusively affects males but is transmitted by female carriers.

The terms “sample,” “test sample,” “sample from a subject,” and “patient sample” are synonymous and refer to any bodily substance obtained from a subject, typically a mammalian subject, and preferably a human subject. The sample may be blood (e.g., whole blood), a blood component (e.g., serum, plasma, red blood cells, leukocytes, or monocytes), a tissue, urine, amniotic fluid, cerebrospinal fluid, placental cells, or endothelial cells. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. Methods for obtaining different types of biological samples from humans are well known in the art and may be used in the connection with the methods described herein. In some embodiments, the sample is a serum sample. A sample may be obtained from a subject suffering from any motor neuron disease, or a subject suspected of suffering from any motor neuron disease, such as those described above or otherwise known in the art. In some embodiments, the subject suffers from or is suspected of suffering from ALS. In some embodiments, a sample is obtained from a subject suffering from or suspected of suffering from upper motor neuron-dominant ALS.

As used herein, the terms “treatment,” “treating,” “therapy,” and the like refer to obtaining a desired pharmacologic and/or physiologic effect. Preferably, the effect is therapeutic, i.e., the effect partially or completely cures a disease and/or adverse symptom attributable to the disease.

### Biomarker Detection and Quantification

The methods described herein involve detecting and quantifying the amount of at least two proteins in a serum sample obtained from a subject suffering from a motor neuron disease, or a subject suspected of suffering from a motor neuron disease. Why not being bound to any particular theory, it is believed that the amount of the at least two proteins, when compared to healthy controls, may inform clinicians regarding the diagnosis, severity, and upper motor neuron involvement of a particular motor neuron disease (e.g., ALS). Furthermore, the presence of certain proteins described herein in the blood may provide information as to the cause(s) of neurodegenerations. For example, an increase or decrease in the at least two proteins in a subject's sample as compared to healthy controls may be indicative of the presence of ALS or other motor neuron disease in the subject. A change in the levels of two or more proteins over a duration of time may indicate a higher or lower degree of disease severity, and/or the involvement of upper motor neurons.

Any suitable combination of proteins may be detected and quantified in accordance with the described methods. Suitable proteins include, but are not limited to, proteins that are expressed in motor neurons. Any suitable number or proteins greater than or equal to two may be detected and quantified. In some embodiments, the method comprises detecting and quantifying 2-10 proteins (i.e., 2, 3, 4, 5, 6, 7, 8, 9, or 10 proteins), 10-20 proteins (e.g., 10, 11, 12, 13, 14, 15, 16, 17, 18, 19, or 20 proteins), 20-30 proteins (e.g., 21, 22, 23, 24, 25, 26, 27, 28, 29, or 30 proteins), 30-40 proteins (e.g., 31, 32, 33, 34, 35, 36, 37, 38, 39, or 40 proteins), or at least 40 proteins (e.g., 40, 50, 60, or 70 or more proteins).

In some embodiments, the two or more proteins are selected from apolipoprotein C3 (APOC3), apolipoprotein F (APOF), complement C8 beta chain (C8B), complement C8 gamma chain (C8G), immunoglobulin heavy constant gamma 3 (IGHG3), inter-alpha-trypsin inhibitor heavy chain 3 (ITIH3), quiescin sulfhydryl oxidase 1 (QSOX1), SERPINA10, SERPINA5, von Willebrand factor (VWF), apolipoprotein A2 (APOA2), apolipoprotein A4 (APOA4), apolipoprotein D (APOD), apolipoprotein L1 (APOL1), complement C4-B (C4B), C-type lectin domain family 3 member B (CLEC3B), clusterin (CLU), amyloid P component, serum (APCS), butyrylcholinesterase (BCHE), complement C1R (C1R), complement factor H (CFH), glycoprotein 1b platelet subunit alpha (GP1BA), protein S (PROS1), SERPINA4, selenoprotein P (SEPP1), alpha-2-Macroglobulin (A2M), angiotensinogen (AGT), complement C1r subcomponent like (C1RL), cluster of differentiation 14 (CD14), ficolin-2 (FCN2), SERPINA1, SERPINF2, apolipoprotein A1 (APOA1), and insulin like growth factor binding protein 3 (IGFBP3).

Any combination of two or more, including all, of the aforementioned proteins may be detected and quantified in accordance with the described methods. For example, in certain embodiments, the disclosure provides a method which comprises detecting and quantifying the proteins apolipoprotein C3 (APOC3), apolipoprotein F (APOF), complement C8 beta chain (C8B), complement C8 gamma chain (C8G), immunoglobulin heavy constant gamma 3 (IGHG3), inter-alpha-trypsin inhibitor heavy chain 3 (ITIH3), quiescin sulfhydryl oxidase 1 (QSOX1), SERPINA10, SERPINA5, and von Willebrand factor (VWF). In other embodiments, the disclosure provides a method which comprises detecting and quantifying the proteins apolipoprotein A2 (APOA2), apolipoprotein A4 (APOA4), apolipoprotein D (APOD), apolipoprotein L1 (APOL1), complement C4-B (C4B), C-type lectin domain family 3 member B (CLEC3B), and clusterin (CLU). In other embodiments, the disclosure provides a method in which the proteins amyloid P component, serum (APCS), butyrylcholinesterase (BCHE), complement C1R (C1R), complement factor H (CFH), glycoprotein 1b platelet subunit alpha (GP1BA), protein S (PROS1), SERPINA4, and SERPINA5 are detected and quantified. In yet other embodiments, the disclosure provides a method in which the proteins amyloid P component, serum (APCS), butyrylcholinesterase (BCHE), complement C1R (CIR), complement factor H (CFH), glycoprotein 1b platelet subunit alpha (GP1BA), protein S (PROS1), SERPINA4, and SERPINA5 are detected and quantified. The methods described herein also encompass detecting and quantifying all of the proteins APOC3, APOF, C8B, C8G, IGHG3, ITIH3, QSOX1, SERPINA10, SERPINA5, VWF, APOA2, APOA4, APOD, APOL1, C4B, CLEC3B, CLU, APCS, BCHE, CIR, CFH, GP1BA, PROS1, SERPINA4, SEPP1, A2M, AGT, C1RL, CD14, FCN2, SERPINA1, SERPINF2, APOA1, and IGFBP3.

The two or more proteins may be detected and quantified in the serum sample using any suitable method for protein detection and/or quantification known in the art. Such methods include, but are not limited to, immunoassays (e.g., enzyme linked-immunosorbent assay (ELISA)), protein immunoprecipitation, immunoelectrophoresis, chemical analysis, SDS-PAGE and Western blot analysis, protein immunostaining, electrophoresis analysis, competitive binding assays, functional protein assays, protein microarray, or chromatography or spectrometry methods (e.g., high-performance liquid chromatography (HPLC), mass spectrometry, liquid chromatography-mass spectrometry (LC/MS), capillary electrophoresis (CE)-MS, or any separating front end coupled with MS detection and quantification) (see, e.g., Salvatore Sechi, Quantitative Proteomics by Mass Spectrometry (Methods in Molecular Biology) 2nd ed. 2016 Edition, Humana Press (New York, N.Y., 2009); Daniel Martins-de-Souza, Shotgun Proteomics: Methods and Protocols 2014 edition, Humana Press (New York, N.Y., 2014); Jörg Reinders and Albert Sickmann, Proteomics: Methods and Protocols (Methods in Molecular Biology) 2009 edition, Humana Press (New York, N.Y., 2009); and Jörg Reinders, Proteomics in Systems Biology: Methods and Protocols (Methods in Molecular Biology) 1^{st} ed. 2016 edition, Humana Press (New York, N.Y., 2009)).

In some embodiments, the two or more proteins may be present in the serum sample at low levels that may not be efficiently detected using conventional methods. In such cases, the two or more proteins may be detected using ultrasensitive methodologies and devices specifically designed for detecting low abundant proteins in a sample. Examples of such methodologies and devices include, but are not limited to, microfluidic analytical systems (such as those described in, e.g., Martel, J. M. and Toner, M., Annu Rev Biomed Eng. 2014 Jul. 11; 16:371-96; Martel et al., Annu Rev Biomed Eng. 2014 Jul. 11; 16:371-96; Malhotra et al., Anal. Chem. 84, 6249-6255 (2012); and U.S. Patent Application Publication 2018/0161775 A1), ultra-sensitive ELISA assays (see, e.g., Schubert et al., Sci Rep, 5, 11034 (2015); doi.org/10.1038/srep11034), and nanoparticle-based systems (see, e.g., Li et al., Biosensors and Bioelectronics, 68: 626-632 (2015)).

Methods and devices for protein detection and quantification are further described in, e.g., Powers, A. D, and S. P. Palecek, Journal of Healthcare Engineering, 3(4): 503-534 (2012), and are available from a variety of commercial sources, any of which may be used in the methods described herein.

Embodiments are described herein for detecting and quantifying the amount of various proteins for detecting, monitoring, treating, etc. motor neuron disease in a subject. However, embodiments described herein may also be performed by detecting/quantifying two or more nucleic acids (e.g., mRNA, cDNA, etc.) encoding the proteins described herein in a sample (e.g., serum sample) from a subject (e.g., suffering from a motor neuron disease). In some embodiments, expression of two or more genes encoding the proteins described herein is monitored and/or quantified. In some embodiments, mRNA encoding two or more genes encoding the proteins described herein is converted into cDNAs (e.g., full-length cDNA, cDNA spanning an exon-exon junction, etc.). In some embodiments, RNA is reverse transcribed into cDNA (e.g., full-length cDNA, cDNA spanning an exon-exon junction, etc.). In some embodiments, two or more cDNAs (e.g., full-length cDNA, cDNA spanning an exon-exon junction, etc.) encoding proteins described herein are provided. In some embodiments, a panel or composition is provided comprising two or more cDNAs (e.g., full-length cDNA, cDNA spanning an exon-exon junction, etc.) encoding two or more proteins described herein (e.g., selected from apolipoprotein C3 (APOC3), apolipoprotein F (APOF), complement C8 beta chain (C8B), complement C8 gamma chain (C8G), immunoglobulin heavy constant gamma 3 (IGHG3), inter-alpha-trypsin inhibitor heavy chain 3 (ITIH3), quiescin sulfhydryl oxidase 1 (QSOX1), SERPINA10, SERPINA5, von Willebrand factor (VWF), apolipoprotein A2 (APOA2), apolipoprotein A4 (APOA4), apolipoprotein D (APOD), apolipoprotein L1 (APOL1), complement C4-B (C4B), C-type lectin domain family 3 member B (CLEC3B), clusterin (CLU), amyloid P component, serum (APCS), butyrylcholinesterase (BCHE), complement C1R (C1R), complement factor H (CFH), glycoprotein 1b platelet subunit alpha (GP1BA), protein S (PROS1), SERPINA4, selenoprotein P (SEPP1), alpha-2-Macroglobulin (A2M), angiotensinogen (AGT), complement C1r subcomponent like (C1RL), cluster of differentiation 14 (CD14), ficolin-2 (FCN2), SERPINA1, SERPINF2, apolipoprotein A1 (APOA1), and insulin like growth factor binding protein 3 (IGFBP3)). In some embodiments, a panel or composition comprises two or more cDNAs (e.g., full-length cDNAs, spanning an exon-exon junction, etc.) described herein but not more that 1000 total cDNAs (e.g., 500 or fewer, 200 or fewer, 100 or fewer, 75 or fewer, 50 or fewer, 40 or fewer, 25 or fewer, 10 or fewer).

### Treatment Methods

The disclosure provides a method of treating a subject suffering from a motor neuron disease. The method comprises (a) detecting and quantifying the amount of at least two proteins in a serum sample obtained from a subject suffering from a motor neuron disease, which two or more proteins are selected from apolipoprotein C3 (APOC3), apolipoprotein F (APOF), complement C8 beta chain (C8B), complement C8 gamma chain (C8G), immunoglobulin heavy constant gamma 3 (IGHG3), inter-alpha-trypsin inhibitor heavy chain 3 (ITIH3), quiescin sulfhydryl oxidase 1 (QSOX1), SERPINA10, SERPINA5, von Willebrand factor (VWF), apolipoprotein A2 (APOA2), apolipoprotein A4 (APOA4), apolipoprotein D (APOD), apolipoprotein L1 (APOL1), complement C4-B (C4B), C-type lectin domain family 3 member B (CLEC3B), clusterin (CLU), amyloid P component, serum (APCS), butyrylcholinesterase (BCHE), complement C1R (CIR), complement factor H (CFH), glycoprotein 1 b platelet subunit alpha (GP1BA), protein S (PROS1), SERPINA4, selenoprotein P (SEPP1), alpha-2-Macroglobulin (A2M), angiotensinogen (AGT), complement C1r subcomponent like (C1RL), cluster of differentiation 14 (CD14), ficolin-2 (FCN2), SERPINA1, SERPINF2, apolipoprotein A1 (APOA1), and insulin like growth factor binding protein 3 (IGFBP3), (b) repeating step (a) at specific time points over a duration of time and measuring a change in concentration of the at least two or more proteins in the serum sample over the duration of time, and (c) administering a treatment to the subject based upon the change in concentration of the at least two or more proteins measured in step (b).

The disclosure also provides method of treating a subject suffering from a motor neuron disease, which method comprises: (a) detecting and quantifying the amount of at least two proteins in a serum sample obtained from a subject suffering from a motor neuron disease, which two or more proteins are selected from apolipoprotein C3 (APOC3), apolipoprotein F (APOF), complement C8 beta chain (C8B), complement C8 gamma chain (C8G), immunoglobulin heavy constant gamma 3 (IGHG3), inter-alpha-trypsin inhibitor heavy chain 3 (ITIH3), quiescin sulfhydryl oxidase 1 (QSOX1), SERPINA10, SERPINA5, von Willebrand factor (VWF), apolipoprotein A2 (APOA2), apolipoprotein A4 (APOA4), apolipoprotein D (APOD), apolipoprotein L1 (APOL1), complement C4-B (C4B), C-type lectin domain family 3 member B (CLEC3B), clusterin (CLU), amyloid P component, serum (APCS), butyrylcholinesterase (BCHE), complement C1R (CIR), complement factor H (CFH), glycoprotein 1 b platelet subunit alpha (GP1BA), protein S (PROS1), SERPINA4, selenoprotein P (SEPP), alpha-2-Macroglobulin (A2M), angiotensinogen (AGT), complement C1r subcomponent like (C1RL), cluster of differentiation 14 (CD14), ficolin-2 (FCN2), SERPINA1, SERPINF2, apolipoprotein A1 (APOA1), and insulin like growth factor binding protein 3 (IGFBP3), (b) detecting an increase or decrease of each of the two or more proteins in the sample as compared to the amounts of the same two or more proteins in a control sample (e.g., a sample obtained from a healthy age-matched individual), and diagnosing the subject as having a motor neuron disease; and (c) administering to the subject a treatment for the motor neuron disease.

Descriptions of detecting and quantifying two or more proteins, motor neuron diseases, and components thereof described above also are applicable to those same aspects of the aforementioned methods of treating a subject suffering from a motor neuron disease.

It will be appreciated that, in order to assess a change in concentration of any of the two or more proteins over time, detection and quantification of the two or more proteins may be performed multiples times over a suitable duration of time. Thus, the step of detecting and quantifying the two or more proteins may be repeated any number of times over a particular duration of time. At minimum, the step of detecting and quantifying the two or more proteins is performed twice over a duration of time. In other embodiments, the step of detecting and quantifying the two or more proteins is repeated two or more times over a duration of time (e.g., 3, 4, 5, 6, 7, 8, 9, 10, 15, 20, 25, 30, or more times). The number of times the step of detecting and quantifying the two or more proteins is repeated will depend on several factors, including the length of the duration of time during which the two or more proteins are assessed, the methods used to detect and quantify the proteins, the severity of the disease in the patient, the rate of progression in the patient, and the number of data points desired by the practitioner.

The duration of time during which the step of detecting and quantifying the two or more proteins is repeated may be any suitable length of time that allows for reliable assessment of a change in concentration of each of the two or more proteins in a subject of interest. It will be appreciated that the duration of time chosen will depend on, inter alia, the extent or severity of disease in the subject and the overall health of the subject. Ideally, to determine the rate of disease progression in a patient, detecting and quantifying the two or more proteins is repeated over a the duration of at least 2 weeks and not longer than 6 months (e.g., 3 weeks, 4 weeks, 6 weeks, 8 weeks, 10 weeks, 12 weeks, 2 months, 3 months, 4 months, or 5 months), so that the difference between time points will help determine the overall rate of progression in a patient. As such, the method can distinguish and determine patients with slowly- or rapidly-progressing disease. It will be appreciated that disease progression information is especially important for drug discovery efforts, which require a means to determine whether a particular compound or therapy improves patient outcome. For example, patients with slowly-progressing disease may present false-positive outcomes in response to a specific disease treatment.

Once the rate of disease progression is determined, disease progression may be monitored by continuing to detect and quantify the two or more proteins over a duration of time from about 1 year to about 10 years in length (e.g., 2, 3, 4, 5, 6, 7, 8, or 9 years), and desirably 5 years, in embodiments where disease progression is slow. In embodiments where disease progression is rapid, disease progression may be monitored by continuing to detect and quantify the two or more proteins over a duration of time from about 1 month to about 5 years in length (e.g., about 1 month, 6 months, 1 year, 2 years, 3 years, or 4 years in length), or the duration of the disease. In other embodiments, the duration of time is about 3 to about 5 years.

Each repetition of the step of detecting and quantifying the two or more proteins may be separated by any suitable length of time. The difference between each time point will suggest whether the patient is a fast progressor or a slow progressor and based on this information the other samples will be collected accordingly. In this regard, for example, each repetition of the step of detecting and quantifying the two or more proteins may be separated by about 30 days or one month. In one embodiment, for example, the method may comprise repeating the step of detecting and quantifying the two or more proteins once every month over a duration of time of 5-10 years, or for the duration of the disease.

In order to determine an appropriate course of treatment, in some embodiments the method comprises measuring an increase or decrease in the amounts of the at least two proteins as compared to the amounts in a control sample and/or measuring a change in concentration of the at least two or more proteins in the serum sample of the subject over the duration of time. Changes in the concentration of proteins and the trend of their increase will inform the clinician about the most appropriate treatment option. Likewise, if a patient is involved in a clinical trial, changes in the protein levels will suggest whether treatment is improving neuron health. In this regard, a change in concentration may be measured in at least one of the two or more proteins. Alternatively, a change in concentration may be measured in each of the two or more proteins that are detected and quantified. In other embodiments, it is possible that no change in concentration of at least one of the two proteins is measured over the duration of time. In some embodiments, the method comprises measuring an increase in at least one of the two or more proteins over the duration of time. For example, the method may comprise measuring an increase in each of the two or more detected and quantified proteins over the duration of time. In addition or alternatively, the method may comprise measuring a decrease in at least one of the two or more proteins, either at a single time point or over a duration of time. For example, the method may comprise measuring a decrease in each of the two or more proteins, either at a single time point or over a duration of time. It also will be appreciated that the method may comprise measuring an increase in the concentration of at least one of the two or more detected and quantified proteins, and a decrease in the concentration of at least one of the two or more detected and quantified proteins, either at a single time point or over a duration of time.

An increase or decrease in the concentration of any combination of the above-described proteins may be measured in accordance with the disclosed method. In some embodiments, the method comprises detecting and quantifying the proteins apolipoprotein C3 (APOC3), apolipoprotein F (APOF), complement C8 beta chain (C8B), complement C8 gamma chain (C8G), immunoglobulin heavy constant gamma 3 (IGHG3), inter-alpha-trypsin inhibitor heavy chain 3 (ITIH3), quiescin sulfhydryl oxidase 1 (QSOX1), SERPINA10, SERPINA5, von Willebrand factor (VWF) in the serum sample and measuring an increase in the concentrations of the proteins over the duration of time. While not being bound to any particular theory, it is believed that an increase in the concentration of the aforementioned proteins over a duration of time may be measured in a broad spectrum of ALS patients.

In other embodiments, the method comprises detecting and quantifying two or more of the proteins SEPP1, SERPINA5, Clusterin (CLU), APOC3, C8B, C8G, immunoglobulin heavy constant gamma 3 (IGHG3), ITIH3, QSOX1, SERPINA10, VWF, amyloid P component, serum (APCS), butyrylcholinesterase (BCHE), complement factor H (CFH), glycoprotein 1b platelet subunit alpha (GP1BA), or SERPINA4 in the serum sample, measuring an increase in the concentrations of the proteins over the duration of time, and determining that the subject suffers from upper motor neuron-dominant ALS. When the subject is a human male, the method may further comprise detecting and quantifying the APO4 protein in the serum sample and measuring an increase in the concentrations of APO4 over the duration of time.

In other embodiments, the method comprises detecting and quantifying the proteins apolipoprotein A2 (APOA2), apolipoprotein A4 (APOA4), apolipoprotein D (APOD), apolipoprotein L1 (APOL1), complement C4-B (C4B), C-type lectin domain family 3 member B (CLEC3B), clusterin (CLU), and insulin like growth factor binding protein 3 (IGFBP3) in the serum sample and measuring a decrease in the concentrations of the proteins over the duration of time. While not being bound to any particular theory, it is believed that a decrease in the concentration of the aforementioned proteins over a duration of time may be measured in a broad spectrum of ALS patients.

In other embodiments, the method comprises detecting and quantifying two or more of the proteins APOA2, APOD, APOL1, C4B, CLEC3B, A2M, AGT, APOA2, C1RL, CD14, FCN2, SERPINA1, SERPINF2, or APOA1 in the serum sample, measuring a decrease in the concentrations of the proteins over the duration of time, and determining that the subject suffers from upper motor neuron-dominant ALS. When the subject is a human female, the method may further comprise detecting and quantifying the APO4 protein in the serum sample and measuring a decrease in the concentrations of APO4 over the duration of time.

In other embodiments, the method comprises detecting and quantifying the proteins amyloid P component, serum (APCS), butyrylcholinesterase (BCHE), complement C1R (C1R), complement factor H (CFH), glycoprotein 1 b platelet subunit alpha (GP1BA), protein S (PROS1), SERPINA4, SERPINA5, and insulin like growth factor binding protein 3 (IGFBP3) in the serum sample, measuring an increase in the concentrations of the proteins over the duration of time, and determining that the subject suffers from upper motor neuron-dominant ALS. While not being bound to any particular theory, it is believed that an increase in the concentration of the aforementioned proteins over a duration of time is indicative of upper motor neuron-dominant ALS.

In other embodiments, the method comprises detecting and quantifying the proteins alpha-2-Macroglobulin (A2M), angiotensinogen (AGT), complement C1r subcomponent like (C1RL), cluster of differentiation 14 (CD14), ficolin-2 (FCN2), SERPINA1, SERPINF2, and apolipoprotein A1 (APOA1) in the serum sample, measuring a decrease in the concentrations of the proteins over the duration of time, and determining that the subject suffers from upper motor neuron-dominant ALS. While not being bound to any particular theory, it is believed that a decrease in the concentration of the aforementioned proteins over a duration of time is indicative of upper motor neuron-dominant ALS.

In some embodiments, once a change in concentration (e.g. increase and/or decrease) has been measured in the two or more proteins, either at a single time point or over the duration of time, the method comprises administering a treatment to the subject based upon the change in concentration of the at least two or more proteins. In this manner, a particular course of treatment can be tailored to the subject based on the biomarker expression profile observed. For example, if it is determined that the subject suffers from upper motor neuron-dominant ALS in accordance with the disclosed methods, then one or more particular treatments may be administered to the subject over another particular treatment. Any suitable treatment or combination of treatments may be administered to the subject. Suitable treatments for motor neuron diseases (such as ALS) are known in the art and include, but are not limited to riluzole (RILUTEK® or TEGLUTIK@), edaravone (RADICAVA®), nusinersen (SPINRAZA®), muscle relaxants, botulinum toxin, amitriptyline, glycopyolate, atropine, dextromethorphan, quinidine, anticonvulsants, nonsteroidal anti-inflammatory drugs (NSAIDs), antidepressants, morphine, opiates, physical therapy, occupational therapy, speech therapy, and psychotherapy. Treatment options for motor neuron diseases are further described in, e.g., Yacila et al., Curr Med Chem., 21(31): 3583-93 (2014); Musaro, A., FEBS J., 280(17): 4315-2 (2013); Simon et al., Intern Med J.; 45(10): 1005-13 (2015); Mitsumoto, H., Amyotrophic Lateral Sclerosis: A Guide for Patients and Families, Third Edition, Demos Health (2009); Bedlack, R. S. and H. Mitsumoto (eds), Amyotrophic Lateral Sclerosis: A Patient Care Guide for Clinicians, 1^{st} Edition, Demos Medical (2012); and Boulis N. M. et al. (eds.), Molecular and Cellular Therapies for Motor Neuron Disease, 1^{st} Edition, Academic Press (2017).

In addition to or alternative to treatment, the methods described herein may be used to monitor a subject during and after any course of treatment (such as the treatments disclosed herein). In yet another alternative, the methods described herein may be used to monitor the progression of a motor neuron disease, such as by assessing whether the subject has a moderate or severe motor neuron disease, and if the severity of such disease increases or decreases over a duration of time. In other embodiments, the methods described herein may be used to aid in the diagnosis, prognosis, risk stratification, and evaluation of whether a subject has a motor neuron disease, such as ALS. More specifically, any combination of the biomarkers described herein can be used in diagnostic tests to determine, qualify, and/or assess motor neuron disease status in a subject or patient. In this regard, for example, determining whether a subject has a motor neuron disease can include measuring or detecting two or more of the above-described protein biomarkers and integrating that information with other information (e.g., clinical assessment data, reference levels for each of two or more protein biomarkers), to determine that the subject is more likely than not to have a motor neuron disease, and in some embodiments, the type of motor neuron disease may also be determined.

### ALS Protein Biomarkers

The following protein biomarkers may be detected and quantified in accordance with the methods described herein. This foregoing list of proteins is merely exemplary and the disclosure is not limited to these specific proteins.

Apolipoprotein C3 (APOC3) is a 10852 Da protein comprising 99 amino acids. APOC3 is a component of triglyceride (TG)-rich lipoproteins (TRLs) including very low density lipoproteins (VLDL), high density lipoproteins (HDL) and chylomicrons. APOC3 plays a role in role in the metabolism of these TRLs through multiple modes. APOC3 has been shown to promote the secretion of VLDL1, inhibit lipoprotein lipase enzyme activity, and delay catabolism of TRL remnants. Mutations in the APOC3 gene are associated with low plasma triglyceride levels, reduced risk of ischemic cardiovascular disease, and hyperalphalipoproteinemia, which is characterized by elevated levels of high density lipoprotein (HDL) and HDL cholesterol in human patients. Extracellularly, APOC3 attenuates hydrolysis and clearance of TRLs, and impairs the lipolysis of TRLs by inhibiting lipoprotein lipase and the hepatic uptake of TRLs by remnant receptors. APOC3 is comprised of several curved helices connected via semiflexible hinges, which enables the protein to wrap tightly around a curved micelle surface and easily adapt to the different diameters of its natural binding partners.

Apolipoprotein F (APOF) is a 35399 Da protein comprising 326 amino acids and is one of the minor apolipoproteins found in plasma. APOF forms complexes with lipoproteins and may be involved in transport and/or esterification of cholesterol. In particular, APOF associates with LDL, and to a lesser degree with VLDL, Apo-AI and Apo-AII. APOF also inhibits cholesteryl ester transfer protein (CETP) activity.

Complement C8 beta chain (C8B) protein is a 67047 Da protein comprising 591 amino acids and a heterotrimer of 3 chains: alpha, beta and gamma. Complement C8 gamma chain (C8G) protein C8B is a22277 Da protein comprising 202 amino acids. C8B and C8G, along with complement C8 alpha chain protein (C8A) form the complement component 8 (C8) protein. C8 is one component of the membrane attack complex (MAC), which mediates cell lysis, and C8 initiates membrane penetration of the complex. In particular, C8B mediates the interaction of C8 with the C5b-7 membrane attack complex precursor, while C8G plays a role in the activation of complement C4 and C2 proteins, as well as complement and coagulation cascades. In humans, C8B deficiency is associated with Complement Component 8 Deficiency (type II), Immunodeficiency Due to A Late Component of Complement Deficiency, and increased risk of meningococcal infections. Diseases associated with C8G deficiency include, for example, Immunodeficiency Due to a Late Component of Complement Deficiency and acute Salpingo-Oophoritis.

Immunoglobulin heavy constant gamma 3 (IGHG3) is a 41287 Da protein comprising 377 amino acids, which is involved in the activation of complement C4 and C2 proteins. Diseases associated with IGHG3 include, for example, Heavy Chain Disease and Gamma Heavy Chain Disease.

Inter-alpha-trypsin inhibitor heavy chain 3 (ITIH3) is a 99849 Da protein comprising 890 amino acids. ITIH3 is the heavy chain subunit of the pre-alpha-trypsin inhibitor complex, which may stabilize the extracellular matrix through its ability to bind hyaluronic acid. ITIH3 may act as a carrier of hyaluronan in serum or as a binding protein between hyaluronan and other matrix proteins to regulate the localization, synthesis, and degradation of hyaluronan, which are essential to cells undergoing biological processes. In an examination of serum samples from ALS patients, high levels of ITIH3 were observed in 75% of ALS patients when compared to healthy age-matched controls. ITIH3 increased over time in all ALS patients, but this increase was prominent in only 46% of ALS patients with UMN loss. Thus, in some embodiments, ITIH3 may be used as a biomarker for ALS disease progression.

Quiescin sulfhydryl oxidase 1 (QSOX1) is an 82578 Da protein comprising 747 amino acids. Expression of the gene encoding QSOX1 is induced as fibroblasts begin to exit the proliferative cycle and enter quiescence, suggesting that QSOX1 plays an important role in fibroblast growth regulation. QSOX1 catalyzes the oxidation of sulfhydryl groups in peptide and protein thiols to disulfides with the reduction of oxygen to hydrogen peroxide and may contribute to disulfide bond formation in a variety of secreted proteins.

SERPIN10 is a 50707 Da protein comprising 444 amino acids. SERPIN10 belongs to the SERPIN protein family and is predominantly expressed in the liver and secreted in plasma. SERPIN10 inhibits the activity of coagulation factors Xa and XIa in the presence of protein Z, calcium, and phospholipid. Diseases associated with SERPIN10 include, for example, protein Z deficiency and thrombosis.

Von Willebrand Factor (VWF) is a glycoprotein of over 300 kDa and 2813 amino acids that is involved in homeostasis. In particular, VWF promotes adhesion of platelets to the sites of vascular injury by forming a molecular bridge between sub-endothelial collagen matrix and platelet-surface receptor complex GPIb-IX-V. VWF also acts as a chaperone for coagulation factor VIII, delivering it to the site of injury, stabilizing its heterodimeric structure, and protecting it from premature clearance from plasma. Deficiency in VWF is associated with, for example, Von Willebrand Disease, Types I and II.

Apolipoprotein A2 (APOA2) is protein of over 11 kDa and 100 amino acids and is the second most abundant protein of the high density lipoprotein particles. APOA2 is found in plasma as a monomer, homodimer, or heterodimer with apolipoprotein D. Defects may result in apolipoprotein A-II deficiency or hypercholesterolemia. Diseases associated with APOA2 include hypercholesterolemia, APOA2 deficiency, and APOAII amyloidosis.

Apolipoprotein A4 (APOA4) is a protein of over 45 kDa and 396 amino acids. Although its precise function is not known, APOA4 is a potent activator of lecithin-cholesterol acyltransferase in vitro, and is a major component of HDL and chylomicrons. Diseases associated with APOA4 (Apolipoprotein A4) include, for example, carotenemia and familial hypertriglyceridemia, Familial.

Apolipoprotein D (APOD) is a protein of over 21 kDa and 189 amino acids. APOD is a component of high density lipoprotein that has no marked similarity to other apolipoprotein protein sequences, but exhibits a high degree of homology to plasma retinol-binding protein and other members of the alpha 2 microglobulin protein superfamily of carrier proteins. Diseases associated with APOD include, for example, breast cysts and Niemann-Pick disease.

Apolipoprotein L1 (APOL1) is a secreted high density lipoprotein protein of over 43 kDa and 398 amino acids which binds to apolipoprotein A1 (APOA1). APOL1 may play a role in lipid exchange and transport throughout the body, as well as in reverse cholesterol transport from peripheral cells to the liver. Diseases associated with APOL1 include, for example, focal segmental focal segmental glomerulosclerosis 4 and sleeping sickness.

Complement C4 beta chain (C4B) protein a protein of more than 19 kDa and 1744 amino acids and is the basic form of complement factor 4, which is expressed as a single chain precursor which is proteolytically cleaved into a trimer of alpha, beta, and gamma chains prior to secretion. The trimer provides a surface for interaction between an antigen-antibody complex and other complement components. C4 is the non-enzymatic component of the C3 and C5 convertases, and is thus essential for the propagation of the classical complement pathway. C4 covalently binds to immunoglobulins and immune complexes and enhances the solubilization of immune aggregates and the clearance of immune complexes through CR1 on erythrocytes. The C4A isotype is responsible for effective binding to form amide bonds with immune aggregates or protein antigens, while the C4B isotype catalyzes the transacylation of the thioester carbonyl group to form ester bonds with carbohydrate antigens. Diseases associated with C4B include complement component 4B deficiency and Immunodeficiency Due to a Classical Component Pathway Complement Deficiency. In an examination of serum samples from ALS patients, high levels of C4B were observed in 75% of ALS patients when compared to healthy age-matched controls. C4B increased in only 16% of ALS patients over time, but increased in about 50% of ALS patients with prominent UMN loss over time. The increased C4B levels over time in ALS patients with UMN loss was most pronounced in males, as 100% tested exhibited increased C4B over time.

C-type lectin domain family 3 member B (CLEC3B), also known as tetranectin, is a protein of more than 22 kDa and 202 amino acids, which is a trimeric Ca²⁺-binding protein belonging to the family of C-type lectins and is mainly found in plasma and extracellular matrix. Tetranectin binds to plasminogen kringle 4, to sulfated polysaccharides, and to extracellular matrix proteins, enhancing plasminogen activation in the presence of tissue plasminogen activator. Therefore, tetranectin is thought to participate in plasminogen activator-related biological processes such as tissue degradation/remodeling and extracellular proteolysis. It has also been suggested that tetranectin has a potential role in the mineralization process in osteogenesis. In an examination of serum samples from ALS patients, CLEC3B levels were reduced in most patients, as about 72% of tested ALS patients had lower levels than controls. About 83% of ALS patients displayed decreases in CLEC3B levels over time. 80% of ALS patients with prominent UMN involvement also displayed decreased levels of CLEC3B over time.

Clusterin (CLU) is a protein of more than 52 kDa and 449 amino acids, which is a secreted chaperone that can be found in the cell cytosol under some stress conditions. CLU may be involved in several basic biological events such as cell death, tumor progression, and neurodegenerative disorders. Alternate splicing results in both coding and non-coding variants. Diseases associated with CLU include follicular dendritic cell sarcoma and transient neonatal neutropenia.

Insulin like growth factor binding protein 3 (IGFBP3) is a protein of more than 31 kDa and 291 amino acids and is a member of the insulin-like growth factor binding protein (IGFBP) family with an IGFBP domain and a thyroglobulin type-I domain. IGFBP3 forms a ternary complex with insulin-like growth factor acid-labile subunit (IGFALS) and either insulin-like growth factor (IGF) I or II. In this form, it circulates in the plasma, prolonging the half-life of IGFs and altering their interaction with cell surface receptors. Defects in IGFBP3 are associated, for example, insulin-like growth factor I and acid-labile subunit deficiency.

Amyloid P component (APCS) is a 223 amino acid protein which is found in the basement membrane and associated with amyloid deposits. APCS is a member of the pentraxin protein family and can interact with DNA and histones and may scavenge nuclear material released from damaged circulating cells. APCS may also function as a calcium-dependent lectin, as it binds two calcium ions per subunit. APCS also binds to carbohydrate, unfolded proteins, and virion particles. APCS expression is reported in acute-phase response, innate immune response, glycoprotein metabolic process, and monocyte differentiation, and is typically expressed in serum and urine. In an examination of serum samples from ALS patients, high levels of APCS levels were observed in over 75% of ALS patient samples as compared to healthy age-matched controls. APCS levels showed an increase over time in over 30% of ALS patients in general, but in 100% of ALS patients with prominent UMN involvement.

Butyrylcholinesterase (BCHE) is a protein of more than 68 kDa and 608 amino acids, which is a member of the type-B carboxylesterase/lipase family of proteins. BCHE exhibits broad substrate specificity and is involved in the detoxification of poisons, including organophosphate nerve agents and pesticides, and the metabolism of drugs including cocaine, heroin, and aspirin. Humans homozygous for certain mutations in this gene exhibit prolonged apnea after administration of the muscle relaxant succinylcholine. Other diseases associated with BCHE include pseudocholinesterase deficiency and cocaine intoxication. In an examination of serum samples from ALS patients, high levels of BCHE were observed in 72% of ALS patients when compared to healthy age-matched controls. Half of ALS patients displayed an increase in BCHE over time. 63% of ALS patients with UMN involvement displayed an increase in BCHE over time.

Complement C1R (C1R) protein is a serine protease of over 80 kDa and 705 amino acids that combines with C1q and C1s to form C1, the first component of the classical pathway of the complement system. C1R is associated with Ehlers-Danlos syndrome, Periodontal Type, 1.

Complement factor H (CFH) is a protein of over 139 kDa and 1231 amino acids, which is a member of the Regulator of Complement Activation (RCA) gene cluster. CFH is secreted into the bloodstream and has an essential role in the regulation of complement activation, and restricting this innate defense mechanism to microbial infections. Mutations in the gene encoding CFH have been associated with hemolytic-uremic syndrome (HUS) and chronic hypocomplementemic nephropathy. In an examination of serum samples from ALS patients, high levels of CFH were observed in 47% of ALS patients when compared to healthy age-matched controls. 63% of ALS patients with prominent UMN loss displayed increased levels of CFH over time. High levels of CFH in ALS patients may indicate the presence of neuroimmune modulation and activation of the complement system.

Glycoprotein 1b platelet subunit alpha (GP1BA) is a protein of more than 71 kDa and 652 amino acids, which is the alpha chain subunit of lycoprotein 1b (GP1B). GP1B is a platelet surface membrane glycoprotein having an alpha chain and a beta chain linked by disulfide bonds. GB1B functions as a receptor for von Willebrand factor (VWF), and the complete receptor complex involves noncovalent association of the alpha and beta subunits with platelet glycoprotein IX and platelet glycoprotein V. The binding of the GP1B-IX-V complex to VWF facilitates initial platelet adhesion to vascular subendothelium after vascular injury, and also initiates signaling events within the platelet that lead to enhanced platelet activation, thrombosis, and hemostasis. Mutations in the GP1BA gene are associated with Bernard-Soulier syndromes and platelet-type von Willebrand disease, and the coding region of the gene contains a polymorphic variable number tandem repeat (VNTR) domain that is associated with susceptibility to nonarteritic anterior ischemic optic neuropathy. In an examination of serum samples from ALS patients, high levels of GP1BA were observed in 73% of ALS patients when compared to healthy age-matched controls. About 33% of ALS patients exhibited an increase in GP1BA over time, but 100% of ALS patients with UMN involvement showed an increased level over time.

Protein S (PROS1) is a protein of greater than 75 kDa and 676 amino acids and is a vitamin K-dependent plasma protein that functions as a cofactor for the anticoagulant protease, activated protein C (APC) to inhibit blood coagulation. PROS1 is found in plasma in both a free, functionally active form and also in an inactive form complexed with C4b-binding protein. Mutations in the gene encoding PROS1 result in autosomal dominant hereditary thrombophilia.

SERPINA4 and SERPINA5 are members of the serpin protein family. SERPINA4 is a protein of more than 48 kDa and 427 amino acids, which inhibits human amidolytic and kininogenase activities of tissue kallikrein. Inhibition is achieved by the formation of an equimolar, heat- and SDS-stable complex between the SERPINA4 and the tissue kallikrein, and generation of a small C-terminal fragment of the SERPINA4 due to cleavage at the reactive site by tissue kallikrein. SERPINA5 is a glycoprotein of more than 45 kDa and 406 amino acids that can inhibit several serine proteases, including protein C and various plasminogen activators and kallikreins, and thus plays diverse roles in hemostasis and thrombosis in multiple organs. SERPINA5 deficiency is associated with periapical periodontitis. As demonstrated below, high SERPINA5 levels have been observed in ALS patients as compared to healthy controls, and this increase is more prominent in males (80% of cases showed increase in male population). In an examination of serum samples from ALS patients, high levels of SERPINA4 were observed in only 38% of ALS patients when compared to healthy age-matched controls. However, in ALS patients with prominent UMN loss about 63% showed increased levels of SERPINA4 over time. SERPINA4 is an indicator of oxidative stress and inflammation, which may contribute to the development and progression of ALS.

Selenoprotein P (SEPP1) is a protein of greater than 43 kDa and 381 amino acids that is predominantly expressed in the liver and secreted into the plasma. SEPP1 contains multiple selenocysteine (Sec) residues per polypeptide (10 in human), and accounts for most of the selenium in plasma. SEPP1 has been implicated as an extracellular antioxidant, and in the transport of selenium to extra-hepatic tissues via apolipoprotein E receptor-2 (apoER2). Mice lacking the SEPP1 gene exhibit neurological dysfunction, suggesting its importance in normal brain function.

Alpha-2-Macroglobulin (A2M) is a protein of more than 160 kDa and 1474 amino acids and is a protease inhibitor and cytokine transporter. A2M uses a bait-and-trap mechanism to inhibit a broad spectrum of proteases, including trypsin, thrombin, and collagenase. A2M can also inhibit inflammatory cytokines, disrupting inflammatory cascades. Mutations in the A2M gene cause alpha-2-macroglobulin deficiency, and A2M deficiency is implicated in Alzheimer's disease (AD) due to its ability to mediate the clearance and degradation of A-beta, the major component of beta-amyloid deposits.

Angiotensinogen (AGT) is a protein of greater than 43 kDa and 485 amino acids, which is expressed in the liver and is cleaved by the enzyme renin in response to lowered blood pressure. The resulting product, angiotensin I, is then cleaved by angiotensin converting enzyme (ACE) to generate the physiologically active enzyme angiotensin II. The protein is involved in maintaining blood pressure and in the pathogenesis of essential hypertension and preeclampsia. Mutations in the AGT gene are associated with susceptibility to essential hypertension, and can cause renal tubular dysgenesis, a severe disorder of renal tubular development. Defects in the AGT gene also are associated with non-familial structural atrial fibrillation and inflammatory bowel disease.

Complement C1r subcomponent like (C1RL) is a protein of over 53 kDa and 487 amino acids, which mediates the proteolytic cleavage of HP/haptoglobin in the endoplasmic reticulum. Diseases associated with C1RL include ovary adenocarcinoma. In an examination of serum samples from ALS patients, high levels of C1RL were observed in over 80% of ALS patient samples as compared to healthy age-matched controls.

Cluster of differentiation 14 (CD14) is a surface antigen of over 40 kDa and 375 amino acids that is preferentially expressed on monocytes/macrophages. CD14 cooperates with other proteins to mediate the innate immune response to bacterial lipopolysaccharide. Diseases associated with CD14 include Mycobacterium chelonae and croup.

Ficolin-2 (FCN2) is a protein of 34 kDa and 313 amino acids which belongs to the ficolin family of proteins. This family is characterized by the presence of a leader peptide, a short N-terminal segment, followed by a collagen-like region and a C-terminal fibrinogen-like domain. This FCN2 gen is predominantly expressed in the liver, and has been shown to have carbohydrate binding and opsonic activities. FCN2 is associated with adenoiditis and necrotizing soft tissue infection. In an examination of serum samples from ALS patients, high levels of FCN2 were observed in 64% of ALS patients when compared to healthy age-matched controls. FCN2 progressively declined in all tested ALS patients with prominent UMN involvement.

SERPINA1 and SERPINF2 are both members of the serpin family of serine protease inhibitors. SERPINA1 is a protein of more than 46 kDa and 418 amino acids which targets elastase, plasmin, thrombin, trypsin, chymotrypsin, and plasminogen activator. Defects in the gene encoding SERPINA1 can cause emphysema or liver disease. Defects in SERPINA1 also are associated with alpha-1-antitrypsin deficiency and hemorrhagic disease due to alpha-1-antitrypsin, Pittsburgh mutation. SERPINF2 is a protein of more than 54 kDa and 491 amino acids and is a major inhibitor of plasmin, which degrades fibrin and various other proteins. Consequently, the proper function of SERPINF2 has a major role in regulating the blood clotting pathway. Mutations in the SERPINF2 gene result in alpha-2-plasmin inhibitor deficiency, which is characterized by severe hemorrhagic diathesis.

Apolipoprotein A1 (APOA1) is a protein of more than 30 kDa and 267 amino acids and is the major protein component of high density lipoprotein (HDL) in plasma. The APOA1 preproprotein is proteolytically processed to generate the mature protein, which promotes cholesterol efflux from tissues to the liver for excretion, and is a cofactor for lecithin cholesterolacyltransferase (LCAT), an enzyme responsible for the formation of most plasma cholesteryl esters. Defects in the APOA1 gene are associated with HDL deficiencies, including Tangier disease, and systemic non-neuropathic amyloidosis. As demonstrated herein, high levels of APOA1 protein have been detected in ALS patients as compared to controls, but APOA1 does not show an increase over time. As such, in some embodiments, APOA1 may be used as a biomarker to distinguish or diagnose ALS.

The following examples further illustrate the invention but, of course, should not be construed as in any way limiting its scope.

### Example 1

Using serum and plasma samples from well-defined ALS patients, blood was collected from the same patients over time (longitudinal samples). Proteomics analysis was conducted using the proteins isolated from the serum samples of patients who were first diagnosed with PLS/HSP but were then determined to have ALS. These patients have primarily upper motor neuron vulnerability and progressive degeneration. The patient characteristics are shown in Table 1.

TABLE 1
Information About Patients

| Sample ID | age | Disease | Gender | Sample # | Bar graph # |
|---|---|---|---|---|---|
| SA/2587 | 48 | | M | Contorl-1 | |
| NA/2580 | 44 | | F | Contorl-2 | |
| AY/2591 | 42 | | F | Contorl-3 | |
| FO/2551 | 41 | | F | Contorl-4 | |
| AK/2891 | 36 | | M | Contorl-5 | |
| KK/2537 | 55 | | F | Contorl-6 | |
| AO/2530 | 23 | | M | Contorl-7 | |
| HC/2553 | 65 | | F | Contorl-8 | |
| 2788 | 67 | | M | Contorl-9 | |
| 3306 | 35 | | F | Contorl-10 | |
| Patient 10a | 68 | ALS (UMN) | F | Patient-1 | F1 |
| Patient 9a | 59 | ALS (UMN) | M | Patient-2 | M1 |
| 1851 | 57 | ALS (UMN) | M | Patient-3 | M2 |
| 1870 | 37 | ALS (UMN) | M | Patient-4 | M3 |
| 1513 | 47 | ALS(UMN) | F | Patient-5 | F2 |
| 1367 | 33 | ALS (UMN) | F | Patient-6 | F3 |
| 1290 | 52 | ALS (UMN) | F | Patient-7 | F4 |
| 1889 | 69 | ALS (UMN) | M | Patient-8 | M4 |
| 1951 | 50 | ALS(UMN) | F | Patient-9 | F5 |
| 2298 | 58 | ALS(UMN) | F | Patient-10 | F6 |
| 2009 | 38 | fALS(UMN) | M | Patient-11 | M5 |
| 1512 | 47 | ALS(UMN) | F | Patient-12 | F7 |
| 1517 | 63 | ALS(UMN) | M | Patient-13 | M6 |
| 1371 | 32 | ALS(UMN) | F | Patient-14 | F8 |
| 2535 | 35 | ALS(UMN) | M | Patient-15 | M7 |
| 1519 | 43 | ALS(UMN) | M | Patient-16 | M8 |
| 2550 | 50 | ALS(UMN) | M | Patient-17 | M9 |
| 3018 | 26 | ALS(UMN) | M | Patient-18 | M10 |
| 1874 | 53 | ALS(UMN) | F | Patient-19 | F9 |
| 1497 | 64 | ALS(UMN) | M | Patient-20 | M11 |
| 3250 | 65 | ALS(UMN) | M | Patient-21 | M12 |

In an initial analysis, APOC3, APOF, C813, C8G, IGHG3, ITEM, QSOX1, SERPINA10, SERPINA5, and VWF showed progressive increase in a broad spectrum of ALS patients. APOA2, APOA4, APOD, APOL1, C411, CLEM, CLU showed progressive decline in a broad spectrum of ALS patients. APCS, BCHE, C1R, CFH, GP1BA, PROS1, SERPINA4, SERPINA5 showed progressive increase in ALS patients with mainly upper motor neuron involvement. SEPP1, A2M, AGT, APOA2, C1RL, CD14, FCN2, SERPINA1, SERPINF2, APOA1 showed progressive decreases in ALS patients with mainly upper motor neuron involvement. IGFBP3 showed progressive decrease in ALS patients, but it was increased in patients with prominent upper motor neuron loss. The slope of these proteins in each patient were similar in all the independent patients investigated. The concentration of these proteins changed in the serum of patients over time, correlating to the disease progression. The change in concentration also allowed for differentiation of patients who developed upper motor neuron involvement from those who did not.

### Example 2

This example demonstrates that levels of SEPP1 protein are increased in the serum of ALS patients with prominent upper motor neuron loss.

Serum samples were collected from the same sporadic ALS (sALS) patients over time (longitudinal samples). Proteomics analysis was conducted using the protein isolated from the serum samples of patients who were first diagnosed with PLS/HSP but were then determined to have ALS.

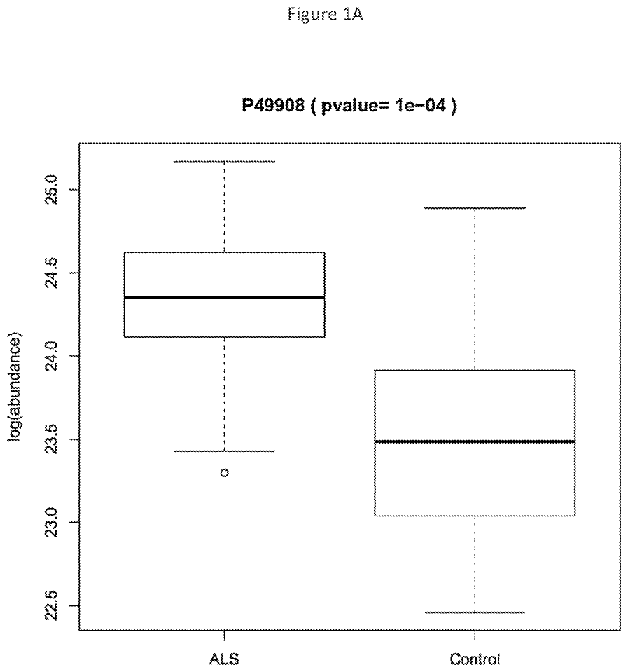

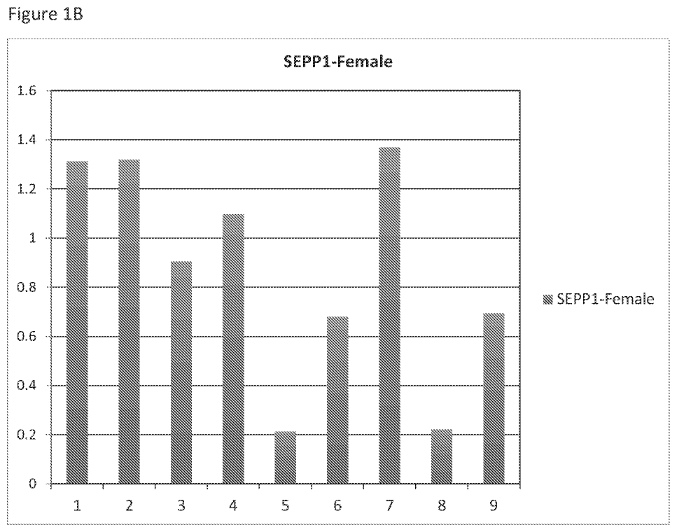

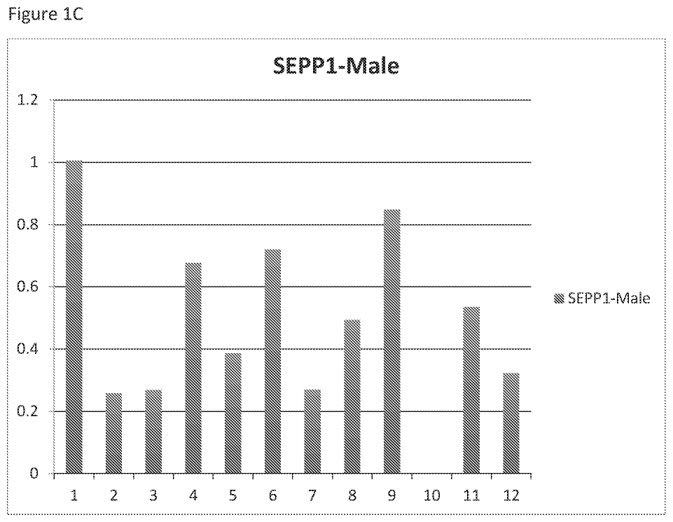

When findings for each time point were averaged for each patient, and fold-change values were determined based on sex and age-matched controls, there was a prominent increase in the levels of SEPP1 in almost all patients, regardless of sex and age, as shown in FIGS. 1A-1C. sALS patients included in the study (n=9 female, n=12 male) with prominent upper motor neuron loss had elevated levels of SEPP1 in their serum when compared to healthy and age-matched controls (n=6 female, n=4 male). These results suggest that an increased level of SEPP1 in the serum is an indicator of upper motor neuron loss in patients. In addition, SEPP1 levels may also serve as a biomarker of disease state, as patients in the early stages of disease progression exhibited lower SEPP1 levels, whereas patients with progressive disease exhibited higher levels of SEPP1.

In addition, an increase of SEPP1 suggests that a patient is struggling to control reactive oxygen species, and that there are increased free radicals in the body. As such, patients with high levels of SEPP1 may be treated with drugs that reduce free radicals and oxidative stress, such as RADICAVA® (edaravone), Coenzyme Q10, and other compounds that reduce the levels of oxidative stress and free radicals. In some embodiments, selenium levels should be monitored in patients, as SEPP1 is one of the major proteins for selenium binding and transport.

### Example 3

This example demonstrates that levels of SERPINA5 protein are increased in the serum of ALS patients with prominent upper motor neuron loss.

Serum samples were collected from the same sporadic ALS (sALS) patients over time (longitudinal samples). Proteomics analysis was conducted using the proteins isolated from the serum samples of patients who were first diagnosed with PLS/HSP but were then determined to have ALS.

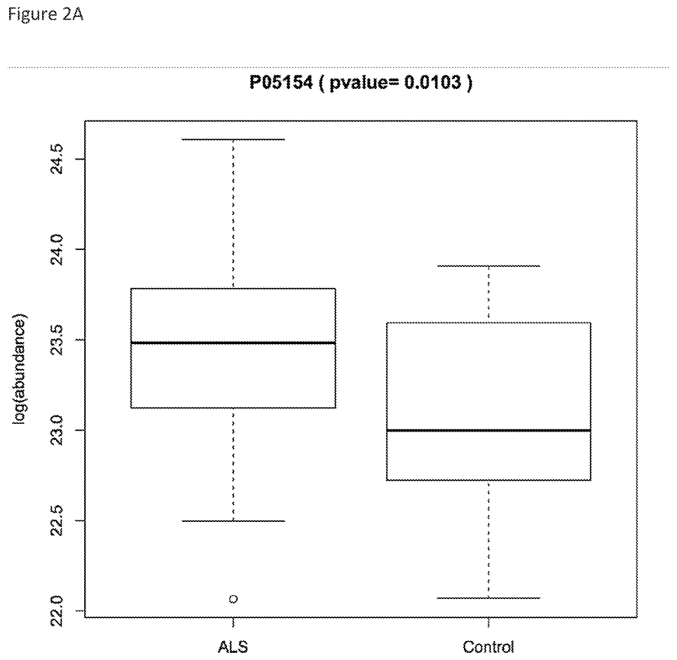

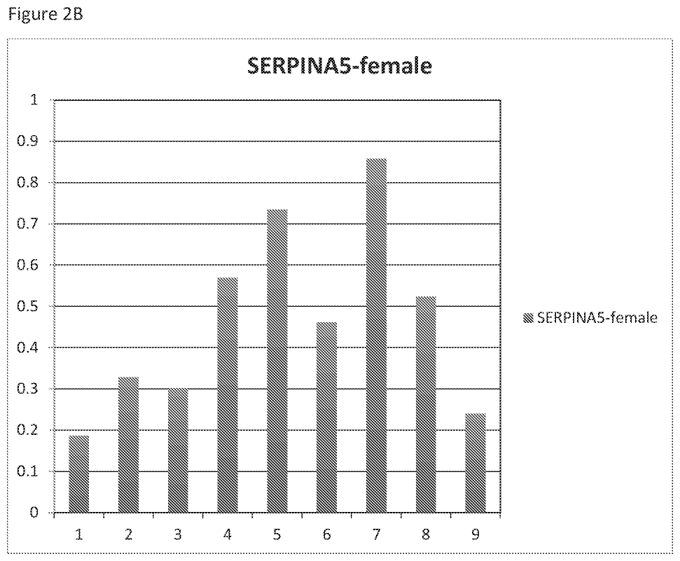

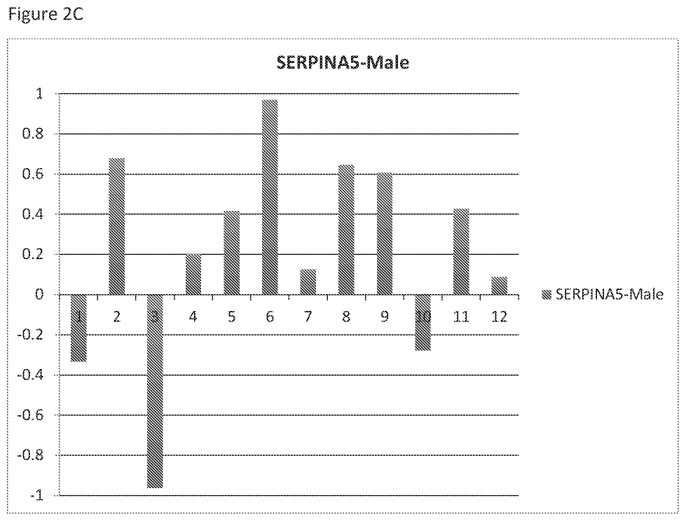

An overall and significant (p=0.0103) increase in the levels of SERPINA5 was observed in the serum of patients with prominent upper motor neuron loss, as shown in FIG. 2A. When males and females were investigated separately, in comparison to age-matched male and female control healthy subjects, a robust increase in the levels of SERPINA5 was observed in both sexes, but especially in females. Indeed, 9 out of 9 tested females displayed increased levels of SERPINA5 when compared to age-matched healthy controls (see FIG. 2B), as compared to 9 out of 12 male patients (3 male patients displayed reduced levels of SERPINA5) (see FIG. 2C). Overall, 18 of 21 patients exhibited higher levels of SERPINA5 when compared to healthy controls.

These results suggest that an increased level of SERPINA5 is an indicator of disease state in patients with upper motor neuron involvement. Patients with high levels of SERPINA5 may have problems with the blood brain barrier, neuroinflammation, and infiltration of cells into the brain parenchyma. As such, in some embodiments, the coagulation profile may be tested in patients to determine potential coagulation defects. Blood lipids may also be profiled, as SERPINA5 also binds and regulates glycosaminoglycans and phospholipids in the blood. Increased lipids in the blood may exacerbate coagulation. In other embodiments, the inflammation state of the patient may also be assessed, and a clinician may treat of detected inflammation, so as to maintain lipid levels within the optimum range and eliminate the possibility of coagulation. Drugs that reduce inflammation, help control levels of lipid in the blood, and/or modulate and inhibit blood coagulation may help patients with increased levels of SERPINA5. For example, in some embodiments, drugs that target the resolution of an inflammatory state, such as minocycline, gammagard, flebogamma, octagam, and valaciclovir may be considered. Likewise, drugs targeting inflammation, such as non-steroidal anti-inflammatory drugs (NSAIDs), ibuprofen, tarenflurbil, salsalate, celecoxib, phenol anti-oxidant resveratrol, etanercept, simvastatin, neflamapimod (vX-745), azeliragon (TTP488), and pioglitazone may help patients with increased levels of SERPINA5.

### Example 4

This example demonstrates that serum levels of APOA4 protein differ between male and female ALS patients.

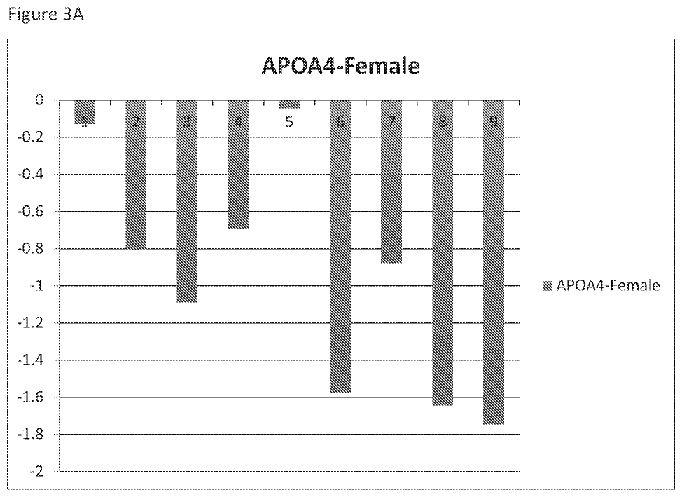

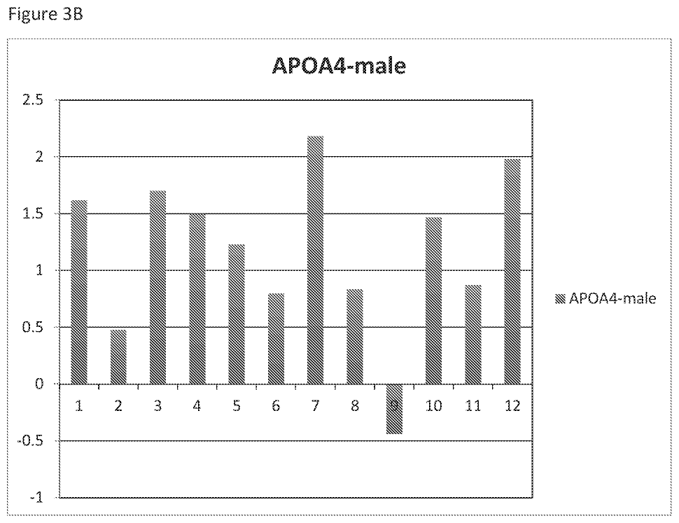

Serum samples were collected from the same sporadic ALS (sALS) patients over time (longitudinal samples). Proteomics analysis was conducted using the proteins isolated from the serum samples of patients who were first diagnosed with PLS/HSP but were then determined to have ALS. Humans respond to dysfunctions in lipid, cholesterol, and fat homeostasis by modulating expression (e.g., increasing or decreasing) APOA4 protein. In ALS patients, there were distinct differences in APOA4 levels between male and female patients. While there was an overall decrease in the levels of APOA4 in female patients with prominent upper motor neuron loss (9 of 9 patients), most of the male patients displayed an increase in the levels of APOA4 (11 of 12 patients), as shown in FIGS. 3A and 3B.

These results may suggest a potential sex difference among patients and that males and females respond differently to problems related to lipid and cholesterol homeostasis. As such, APOA4 levels should be tested in ALS patients to inform clinicians regarding lipid and cholesterol homeostasis problems in female vs. male patients. Patients with APOA4 levels higher or lower than control cases would indicate a defect in lipid homeostasis, and, in some embodiments, could be treated with a drug used to modulate and control cholesterol and lipid homeostasis (e.g., statins, ezetimibe, colesevelam, torcetrapib, avasimibe, implitapide, or niacin).

### Example 5

This example demonstrates that levels of APOF protein are decreased in the serum of ALS patients with prominent upper motor neuron loss.

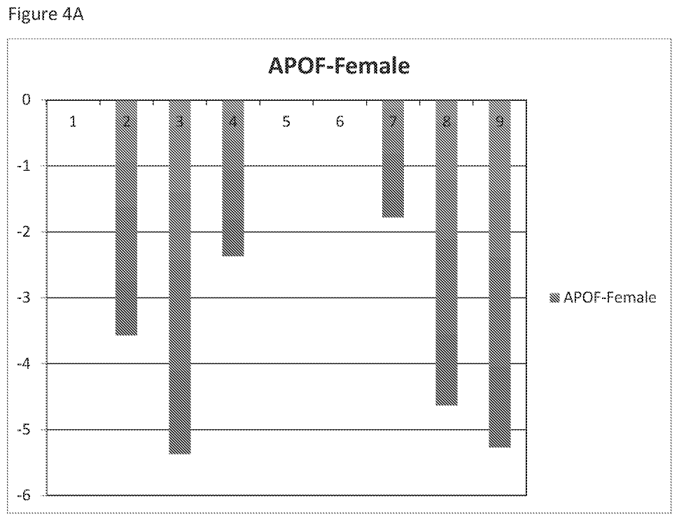

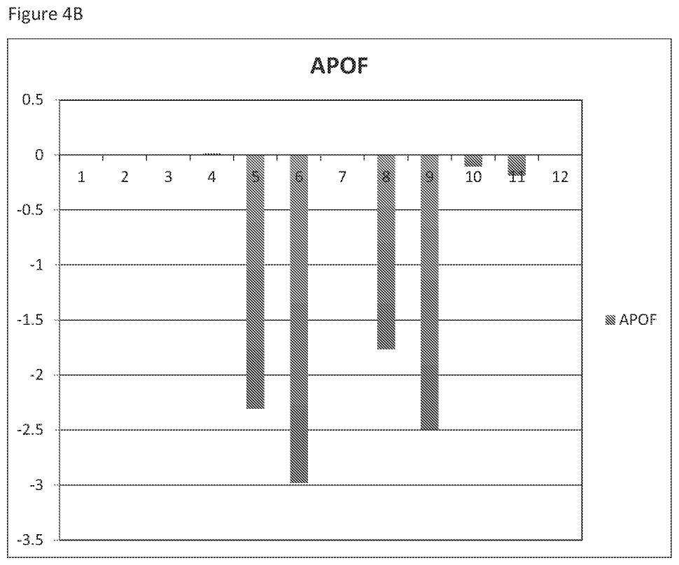

Serum samples were collected from the same sporadic ALS (sALS) patients over time (longitudinal samples). Proteomics analysis was conducted using the proteins isolated from the serum samples of patients who were first diagnosed with PLS/HSP but were then determined to have ALS. A distinct population of motor neuron disease patients with prominent upper motor neuron loss displayed reduced levels of APOF. Six out of 9 female patients had significantly lower levels of APOF when compared to healthy controls, and 3 out of 9 female patients exhibited almost identical APOF levels as compared to control subjects. None of the female patients displayed increased levels of APOF (FIG. 4A). Similarly, in male patients, 6 out of 12 patients exhibited low levels of APOF, and none of the male patients had increased levels of APOF when compared to controls (FIG. 4B).

These results suggest that the levels of APOF may indicate whether altered lipid and cholesterol homeostasis is a contributing factor for upper motor neuron vulnerability in human patients. Thus, in some embodiments, APOF levels may be investigated together with APOA4 to gain a better understanding and assessment of a patient's condition. Reduced levels of APOF and altered levels of APOA4 may suggest that motor neuron degeneration is due in part to problems with lipid and cholesterol homeostasis. In some embodiments, such patients can be further assessed for lipid levels and cholesterol profile and/or treated with a drug used to modulate and control cholesterol and lipid homeostasis (e.g., statins, ezetimibe, colesevelam, torcetrapib, avasimibe, implitapide, or niacin).

### Example 6

This example demonstrates that levels of clusterin (CLU) protein are increased in the serum of ALS patients with prominent upper motor neuron loss.

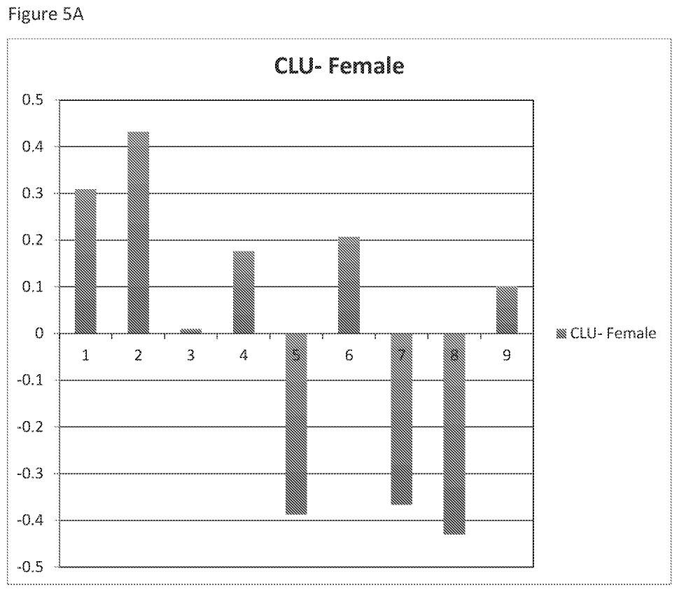

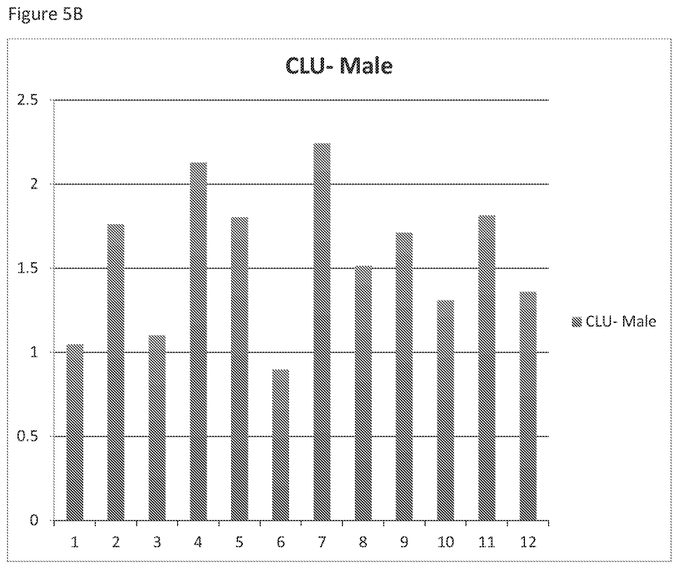

Serum samples were collected from the same sporadic ALS (sALS) patients over time (longitudinal samples). Proteomics analysis was conducted using the proteins isolated from the serum samples of patients who were first diagnosed with PLS/HSP but were then determined to have ALS. CLU levels were increased in all male patients (12 out of 12) with upper motor neuron involvement, increased in 5 out of 9 female patients, and decreased in 3 out of 9 female patients, when compared to age and sex-matched control cases. Overall, CLU levels were increased in 17 of 21 patients with upper motor neuron involvement. The difference in male patients was more prominent than in female patients (FIGS. 5A and 5B).

Because CLU is involved in cellular events that are related to oxidative stress, protein misfolding, and endoplasmic reticulum (ER) stress, increased levels of CLU may indicate that the patient developed ALS as a result of these cellular events. In other words, increased levels of CLU may indicate patients that develop motor neuron vulnerability and progressive degeneration due to problems with oxidative stress, ER stress and protein misfolding. Thus, in some embodiments, ALS patients with increased CLU levels may be monitored for oxidative stress, ER-stress, and protein aggregation defects, and, if necessary, treated with drugs that modulate ER-stress (e.g., valproate, lithium, bortezomib, nelfinavir, atazanavir, arimoclomol, IU1 or IU1-47, geldanamycin, 17-AAG, HSP990, rolipram, or PD169316).

In addition, because CLU also is present in the inner mitochondrial membrane, its detection in serum may suggest problems with mitochondria, especially as the disease progresses. Thus, ALS patients with high levels of CLU may also be considered for treatment with drugs that improve mitochondrial health and integrity, such as NSAIDs, minocycline, KB-R7943, CsA, CoQ10, idebenone, lipoic acid, melatonin, vitamin E, nicotinamide, carnitine, resveratrol, sirtuits, FK 506, deferoxamine, or cardiolipin tetrapeptide MTP-131.

### Example 7

This example demonstrates that levels of complement C1R (C1R) protein are increased in the serum of ALS patients with prominent upper motor neuron loss.

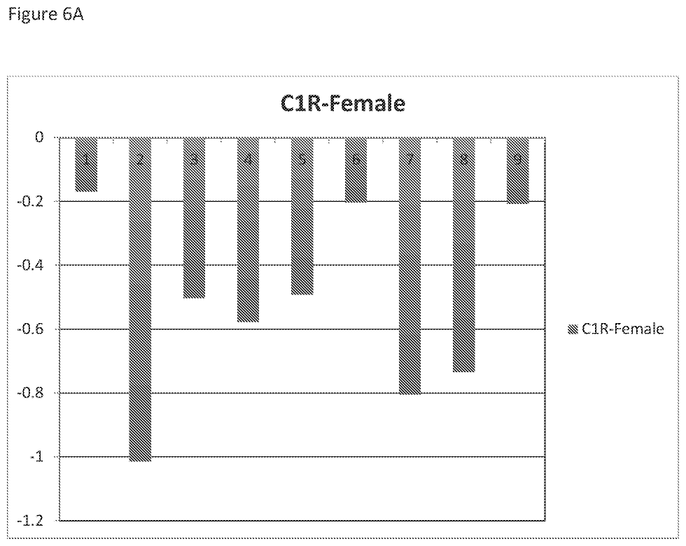

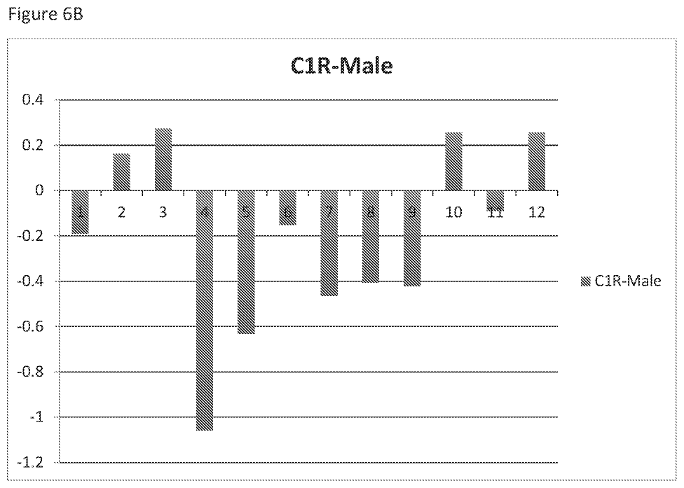

Serum samples were collected from the same sporadic ALS (sALS) patients over time (longitudinal samples). Proteomics analysis was conducted using the proteins isolated from the serum samples of patients who were first diagnosed with PLS/HSP but were then determined to have ALS. Levels of C1R decreased in all females and in 9 out of 12 male patients with prominent upper motor neuron loss (FIGS. 6A and 6B).

Similar to findings with complement factor H (CFH), a C1R imbalance may suggest that neuroimmune modulation is one of the key events that occur in patients with upper motor neuron loss. Thus, in some embodiments, patients may be treated with a broader panel of neuroimmune modulators and/or drugs to control the levels of immune reaction. For example, to modulate microglian activation state, sargramostim, candesartan, telmisartan, GC021109, or azeliragon may be considered. For modulation of eicosanoid signaling, ibuprofen, indomethacin, tarenflurbil, or CHF5074 may be considered. To modulate cytokine signaling, etanercept, thalidomide, neflamapimod, pioglitazone, or rosiglitazone may be considered. To resolve the inflammatory state, minocycline, gammagard, flebogamma, octagam, or valaciclovir may be considered. Drugs targeting inflammation, such as non-steroidal anti-inflammatory drugs (NSAIDs), ibuprofen, tarenflurbil, salsalate, celecoxib, resveratrol, etanercept, simvastatin, neflamapimod (VX-745), azeliragon (TTP488), or pioglitazone may be considered. Clinical strategies used to dampen IL-103 signaling, such as CE-224,535, AZD-9056, GSK-1482160, SGM-1019, JNJ-54175446, JNJ-55308942; OLT-1177; VX-765, VX-740; anakinra, rilonacept, or canakinumab may also be considered.

### Example 8

This example demonstrates that levels of protein S (PROS1) are increased in the serum of ALS patients with prominent upper motor neuron loss.

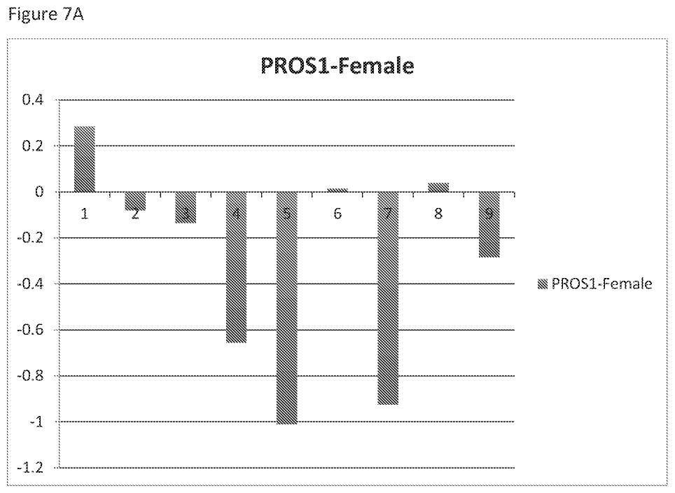

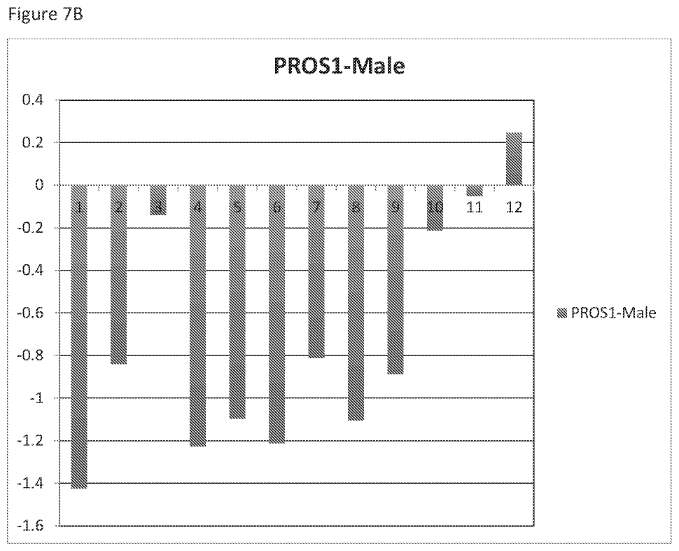

Serum samples were collected from the same sporadic ALS (sALS) patients over time (longitudinal samples). Proteomics analysis was conducted using the proteins isolated from the serum samples of patients who were first diagnosed with PLS/HSP but were then determined to have ALS. Most patients with upper motor neuron involvement displayed low levels of PROS1, when compared to healthy controls. Only 1 out of 9 female patients displayed mild increases in the levels of PROS1, while 6 out of 9 female patients exhibited decreased levels of PROS1 (FIG. 7A). Likewise, most male patients (11 out of 12) had lower levels of PROS1, when compared to age and sex-matched control cases (FIG. 7B). Overall, 17 out of 21 patients exhibited significant reduction in PROS1 levels.

Because PROS1 plays a role regulating apoptosis and systemic inflammation, reduced levels of PROS1 may suggest an increase in the extent of neurodegeneration and inflammation in ALS patients. In addition, because PROS1 is important for the stability of vasculature, reduced levels may also suggest potential problems in blood vessels, leakage of blood brain barrier, and increased infiltration of cells into the brain parenchyma. PROS1 also functions as an anticoagulant, so reduced PROS1 levels may pose potential problems with blood coagulation and activation of the complement system.

Thus, in some embodiments, ALS patients with reduced levels of PROS1 may be assessed for increased immune reaction, levels of blood coagulation factors, and systemic inflammation. Patients may be treated with drugs that reduce blood coagulation, improve the integrity and stability of blood vessel walls, and reduce inflammation. For example, minocycline, gammagard, flebogamma, octagam, or valaciclovir may be administered to reduce inflammation. In addition, drugs that target inflammation, such as non-steroidal anti-inflammatory drugs (NSAIDs), ibuprofen, tarenflurbil, salsalate, celecoxib, phenol anti-oxidant resveratrol, etanercept, simvastatin, neflamapimod (VX-745), azeliragon (TTP488), or pioglitazone may be administered. Anti-coagulation drugs include, for example, apixaban, dabigatran, edoxaban, enoxaparin, heparin, rivaroxaban, and warfarin. In addition, the anti-angiogenic drug bevacizumab may be administered to improve potential damage to the blood vessels in the patients.

### Example 9

This example demonstrates that levels of SERPINF2 protein are decreased in the serum of ALS patients with prominent upper motor neuron loss.

Serum samples were collected from the same sporadic ALS (sALS) patients over time (longitudinal samples). Proteomics analysis was conducted using the proteins isolated from the serum samples of patients who were first diagnosed with PLS/HSP but were then determined to have ALS.

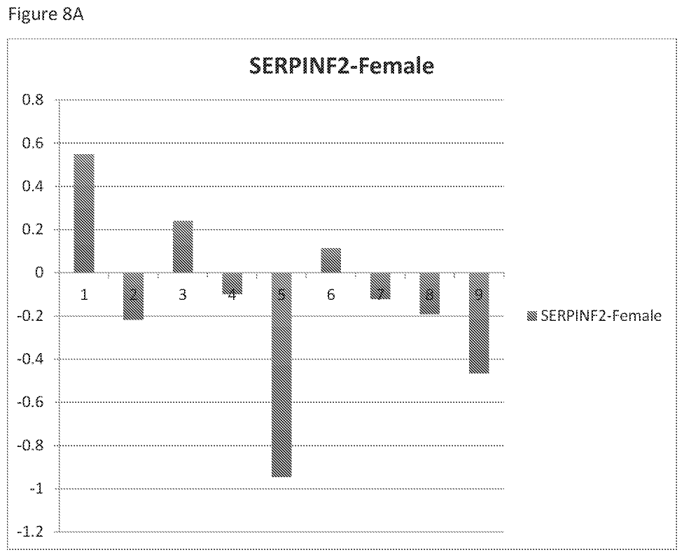

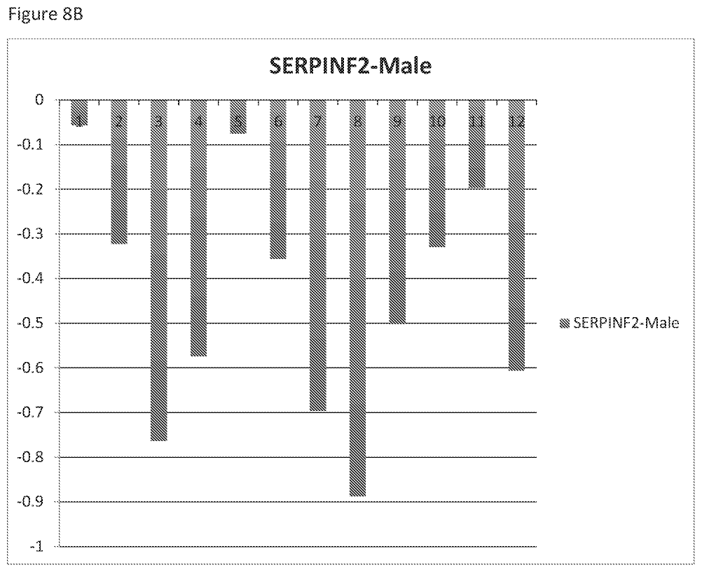

All male patients with upper motor neuron involvement (n=12) displayed reduced levels of SERPINF2. Among all female patients with upper motor neuron involvement (n=9), 6 displayed reduced levels and 3 displayed a slight increase. Overall, 17 of 21 patients had reduced levels of SERPINF2 (FIGS. 8A and 8B).

Reduced levels of SERPINF2 in ALS patients may suggest dysregulation of protease activity. Altered levels of SERPINF2 may also have broad implications for protein-protein interactions, protein accumulation, and ER-stress in neurons.

In some embodiments, the levels of SERPINF2, SERPINA5 and SERPINA4 may be studied together in patients. Their dysregulation in patients, either separately or in combination, may inform the extent of protease inhibition and the canonical pathways that are primarily involved, which may allow more personalized care for each patient.

ALS patients who have decreased levels of SerpinF2 may have cytoarchitectural problems, cell-cell contact problems, a leaky blood brain barrier, and potential problems with protein aggregation. One of the major manifestations of cytoarchitectural defects is problems with axonal transport, because maintaining the cytoarchitectural integrity of the axon is key for proper anterograde and retrograde transport. As such, drugs that improve microtubule stability (e.g., nocodazole, vinblastine, and taxol) may be considered.

### Example 10

This example demonstrates that high levels of two or more of APOA1, SERPINA5, SEPP1, CFH, CLU and QSOX1 proteins, and low levels of APOF protein, are indicative of motor neuron degeneration and ALS.

Serum samples were collected from sporadic ALS (sALS) patients, and proteomics analyses were conducted using the proteins isolated from the serum samples as described above. Protein levels from patient serum samples were compared to protein levels in age and sex matched healthy controls.

APOA1 protein levels were found to be higher in 100% of ALS patients tested (11 out of 11 patients). Given the fact that the tested patients were not related and represented cases of independent distribution, levels of APOA1 in serum samples of patients may be indicative of whether they have ALS or not.

QSOX1 protein levels were found to be higher in 91% of ALS patients tested (10 out of 11 patients). Given the fact that the tested patients were not related and represented cases of independent distribution, levels of QSOX1 in serum samples of patients may be indicative of whether they have ALS or not.

SERPINA5 protein levels were found to be higher in 95.2% of ALS patients tested (20 out of 21 patients). Given the fact that the tested patients were not related and represented cases of independent distribution, levels of SERPINA5 in serum samples of patients may be strongly indicative of ALS.

SEPP1 protein levels were found to be higher in 93.8% of ALS patients tested (30 out of 32 patients). Only 16% of sALS patients showed an increase in SEPP1, but higher percentage of ALS patients with prominent UMN involvement showed increases in SEPP1. Given the fact that the tested patients were not related and represented cases of independent distribution, levels of Sepp1 in serum samples of patients may be strongly indicative of ALS.

CLU protein levels were found to be higher in 84.4% of ALS patients tested (27 out of 32 patients). CLU was increased in 16% of ALS patients, but 70% of ALS patients with prominent UMN involvement exhibited increased CLU levels. Given the fact that the tested patients were not related and represented cases of independent distribution, levels of CLU in serum samples of patients may be strongly indicative of ALS.

APOF protein levels were found to be much lower than the control group in 81.8% of ALS patients tested (9 out of 11 patients). Given the fact that the tested patients were not related and represented cases of independent distribution, reduced levels of APOF in serum of patients may be indicative of whether they have ALS or not.

Overall, these results suggest that high levels of APOA1, SERPINA5, SEPP1, CFH, CLU and QSOX1, are strong indicators of motor neuron degeneration and ALS. Indeed, increased levels of only two of these proteins may be a good indicator of ALS. In contrast, ALS patients are expected to have low levels of APOF.

### Example 11

This example demonstrates that the concentrations of certain proteins described herein increase or decrease over time with motor neuron disease progression.

Blood samples from ALS patients were collected every six months and quantitative protein measurements were performed. Protein levels were compared to those of age and sex matched healthy controls.

In 73.3% of independent ALS cases (22 of 30 patients tested), Serpina5 protein levels increased in ALS patients, and this percentage jumped to 96.2% (25 out of 24 patient tested), when only ALS patients with prominent upper motor neuron involvement were considered. These results suggest that the level of SERPINA5 is a good indicator of ALS and a very strong indicator of upper motor neuron involvement. High levels of SERPINA5 would suggest that the patient has progressive upper motor neuron loss, as levels of SERPINA5 protein increased in serum along with disease progression in ALS patients with prominent upper motor neuron loss.

Likewise, levels of APCS increased in 73.3% of ALS patients over time (22 in 30 independent cases), and this percentage was 87.5% when only ALS patients with prominent upper motor neuron involvement were studied (21 in 24 patients). These results suggest that the level of APCS is a good indicator of ALS and a very strong indicator of upper motor neuron involvement. High levels of APCS would suggest that the patient has progressive upper motor neuron loss, as levels of APCS protein increased in serum along with disease progression in ALS patients with prominent upper motor neuron loss.

CFH levels were high in 70% of ALS patients (21 out of 30 patients tested). CFH levels also were increased in 70.8% of patients with prominent upper motor neuron involvement (17 out of 24 patient tested).

Levels of IGFBP3 were high in 81.8% of ALS patients, with an increase in 63.6% of sporadic ALS patients (7 out of 11). IGFBP3 increased in only 20.8% of all ALS patients, but is increased in 100% of ALS patients with prominent UMN involvement (5 in 5 patients tested). Thus, having increased levels of IGFBP3 is a good indicator of UMN loss.

Levels of GP1BA protein were high in 72.7% of ALS patients, with 63.6% of sporadic ALS patients (7 out of 11) showing increased GP1BA, while an increasing trend was observed in only 20.8% of ALS patients. Decreased levels of GP1BA were observed in about 75% of ALS patients with prominent upper motor neuron involvement (5 in 24 patients). These results suggest that GP1BA is increased in a majority of sporadic ALS patients and decreased in a majority of ALS patients with prominent upper motor neuron loss.

One of the most striking differences was detected with IGHG3. About 63.6% of ALS patients displayed high levels of IGHG3 (7 in 11 patients). These same 63.6% of ALS patients also showed increased levels of IGHG3 over time. However, this was not true for ALS patients with prominent upper motor neuron loss, as only 8.3% of ALS patients with prominent upper motor neuron loss displayed increased levels of IGHG3, whereas about 90% patients had decreased levels of IGHG3. Therefore, these results suggest that the mode of change of IGHG3 over time may be informative about the involvement of upper motor neuron loss in ALS pathology.

APOF levels were low in sporadic ALS patients, and only 18.2% of patients (2 of 11 patients) had higher APOF levels when compared to controls. However, APOF levels showed a dramatic increase over time in ALS patients, with about 63.6% of sporadic ALS patients showing increased APOF levels over time (7 of 11 patients). These results were not observed in ALS patients with prominent upper motor neuron involvement, as only 8.33% of patients displayed an increase over time (2 in 24 patients). Therefore, these results suggest that decreased levels of APOF may be used to identify ALS patients with prominent upper motor neuron loss.

High levels of APOA1 protein were observed in 100% of patients analyzed with both sALS and ALS with prominent UMN involvement (11 out of 11 patients), but the levels decreased, rather than increased, over time in both sALS and ALS patients with prominent UMN involvement.

An increased level of SERPINA5 protein with disease progression may be a strong indicator of prominent upper motor neuron loss, because increased SERPINA5 is observed in 96% of ALS patients with prominent upper motor neuron involvement. Likewise, an increased level of APCS over time may be a strong indicator of progressive and prominent upper motor neuron degeneration in ALS patients, since 87% of ALS patients with prominent upper motor neuron involvement displayed increased APCS over time with disease progression.

In addition to the increase in SERPINA5, CLU, and APCS levels over time in most of ALS patients with prominent upper motor neuron involvement, a gradual decrease in the levels of IGFBP3, GP1BA, APOF and IGHG3 is expected in these patients.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method comprising detecting and quantifying the amount of at least two proteins in a serum sample obtained from a subject suffering from a motor neuron disease, which two or more proteins are selected from apolipoprotein C3 (APOC3), apolipoprotein F (APOF), complement C8 beta chain (C8B), complement C8 gamma chain (C8G), immunoglobulin heavy constant gamma 3 (IGHG3), inter-alpha-trypsin inhibitor heavy chain 3 (ITIH3), quiescin sulfhydryl oxidase 1 (QSOX1), SERPINA10, SERPINA5, von Willebrand factor (VWF), apolipoprotein A2 (APOA2), apolipoprotein A4 (APOA4), apolipoprotein D (APOD), apolipoprotein L1 (APOL1), complement C4-B (C4B), C-type lectin domain family 3 member B (CLEC3B), clusterin (CLU), amyloid P component, serum (APCS), butyrylcholinesterase (BCHE), complement C1R (CIR), complement factor H (CFH), glycoprotein 1 b platelet subunit alpha (GP1BA), protein S (PROS1), SERPINA4, selenoprotein P (SEPP1), alpha-2-Macroglobulin (A2M), angiotensinogen (AGT), complement C1r subcomponent like (C1RL), cluster of differentiation 14 (CD14), ficolin-2 (FCN2), SERPINA1, SERPINF2, apolipoprotein A1 (APOA1), and insulin like growth factor binding protein 3 (IGFBP3).

2. The method of claim 1, wherein the motor neuron disease is amyotrophic lateral sclerosis (ALS), progressive bulbar palsy, pseudobulbar palsy, primary lateral sclerosis (PLS), progressive muscular atrophy, spinal muscular atrophy (SMA), hereditary spastic paraplegia (HSP), or post-polio syndrome (PPS).

3. The method of claim 2, wherein the subject suffers from amyotrophic lateral sclerosis (ALS).

4. The method of claim 3, wherein the subject suffers from upper motor neuron-dominant ALS.

5. The method of any one of claims 1-3, which comprises detecting and quantifying the proteins apolipoprotein C3 (APOC3), apolipoprotein F (APOF), complement C8 beta chain (C8B), complement C8 gamma chain (C8G), immunoglobulin heavy constant gamma 3 (IGHG3), inter-alpha-trypsin inhibitor heavy chain 3 (ITIH3), quiescin sulfhydryl oxidase 1 (QSOX1), SERPINA10, SERPINA5, von Willebrand factor (VWF), apolipoprotein A2 (APOA2), apolipoprotein A4 (APOA4), apolipoprotein D (APOD), apolipoprotein L1 (APOL1), complement C4-B (C4B), C-type lectin domain family 3 member B (CLEC3B), clusterin (CLU), and insulin like growth factor binding protein 3 (IGFBP3) in the serum sample.

6. The method of any one of claims 1-4, which comprises detecting and quantifying the proteins amyloid P component, serum (APCS), butyrylcholinesterase (BCHE), complement CIR (CIR), complement factor H (CFH), glycoprotein 1b platelet subunit alpha (GP1BA), protein S (PROS1), SERPINA4, SERPINA5, selenoprotein P (SEPP1), alpha-2-macroglobulin (A2M), angiotensinogen (AGT), apolipoprotein A2 (APOA2), complement C1r subcomponent like (C1RL), cluster of differentiation 14 (CD14), ficolin-2 (FCN2), SERPINA1, SERPINF2, and apolipoprotein A1 (APOA1) in the serum sample.

7. The method of any one of claims 1-3, which comprises detecting and quantifying the proteins APOC3, APOF, C8B, C8G, IGHG3, ITIH3, QSOX1, SERPINA10, SERPINA5, VWF, APOA2, APOA4, APOD, APOL1, C4B, CLEC3B, CLU, APCS, BCHE, C1R, CFH, GP1BA, PROS1, SERPINA4, SEPP1, A2M, AGT, C1RL, CD14, FCN2, SERPINA1, SERPINF2, APOA1, and IGFBP3 in the serum sample.

8. A method of treating a subject suffering from a motor neuron disease, which method comprises:
(a) detecting and quantifying the amount of at least two proteins in a serum sample obtained from a subject suffering from a motor neuron disease, which two or more proteins are selected from apolipoprotein C3 (APOC3), apolipoprotein F (APOF), complement C8 beta chain (C8B), complement C8 gamma chain (C8G), immunoglobulin heavy constant gamma 3 (IGHG3), inter-alpha-trypsin inhibitor heavy chain 3 (ITIH3), quiescin sulfhydryl oxidase 1 (QSOX1), SERPINA10, SERPINA5, von Willebrand factor (VWF), apolipoprotein A2 (APOA2), apolipoprotein A4 (APOA4), apolipoprotein D (APOD), apolipoprotein L1 (APOL1), complement C4-B (C4B), C-type lectin domain family 3 member B (CLEC3B), clusterin (CLU), amyloid P component, serum (APCS), butyrylcholinesterase (BCHE), complement C1R (C1R), complement factor H (CFH), glycoprotein 1b platelet subunit alpha (GP1BA), protein S (PROS1), SERPINA4, selenoprotein P (SEPP1), alpha-2-Macroglobulin (A2M), angiotensinogen (AGT), complement C1r subcomponent like (C1RL), cluster of differentiation 14 (CD14), ficolin-2 (FCN2), SERPINA1, SERPINF2, apolipoprotein A1 (APOA1), and insulin like growth factor binding protein 3 (IGFBP3),
(b) repeating step (a) at specific time points over a duration of time and measuring a change in concentration of the at least two or more proteins in the serum sample over the duration of time, and
(c) administering a treatment to the subject based upon the change in concentration of the at least two or more proteins measured in step (b).

9. The method of claim 8, wherein the motor neuron disease is amyotrophic lateral sclerosis (ALS), progressive bulbar palsy, pseudobulbar palsy, primary lateral sclerosis (PLS), progressive muscular atrophy, spinal muscular atrophy (SMA), hereditary spastic paraplegia (HSP), or post-polio syndrome (PPS).

10. The method of claim 8 or claim 9, wherein the serum sample is obtained from a subject suffering from amyotrophic lateral sclerosis (ALS).

11. The method of claim 10, which comprises detecting and quantifying the proteins apolipoprotein C3 (APOC3), apolipoprotein F (APOF), complement C8 beta chain (C8B), complement C8 gamma chain (C8G), immunoglobulin heavy constant gamma 3 (IGHG3), inter-alpha-trypsin inhibitor heavy chain 3 (ITIH3), quiescin sulfhydryl oxidase 1 (QSOX1), SERPINA10, SERPINA5, and von Willebrand factor (VWF) in the serum sample and measuring an increase in the concentrations of the proteins over the duration of time.

12. The method of claim 10, which comprises detecting and quantifying the proteins apolipoprotein A2 (APOA2), apolipoprotein A4 (APOA4), apolipoprotein D (APOD), apolipoprotein L1 (APOL1), complement C4-B (C4B), C-type lectin domain family 3 member B (CLEC3B), clusterin (CLU), and insulin like growth factor binding protein 3 (IGFBP3) in the serum sample and measuring a decrease in the concentrations of the proteins over the duration of time.

13. The method of claim 10, which comprises detecting and quantifying the proteins amyloid P component, serum (APCS), butyrylcholinesterase (BCHE), complement C1R (C1R), complement factor H (CFH), glycoprotein 1b platelet subunit alpha (GP1 BA), protein S (PROS1), SERPINA4, SERPINA5, and insulin like growth factor binding protein 3 (IGFBP3) in the serum sample, measuring an increase in the concentrations of the proteins over the duration of time, and determining that the subject suffers from upper motor neuron-dominant ALS.

14. The method of claim 10, which comprises detecting and quantifying two or more of the proteins SEPP1, SERPINA5, Clusterin, APOC3 C8B, C8G, IGHG3, ITIH3, QSOX1, SERPINA10, VWF, APCS, BCHE, CFH, GP1BA, or SERPINA4 in the serum sample, measuring an increase in the concentrations of the proteins over the duration of time, and determining that the subject suffers from upper motor neuron-dominant ALS.

15. The method of claim 14, wherein the subject is a human male and the method further comprises detecting and quantifying the APO4 protein in the serum sample and measuring an increase in the concentrations of APO4 over the duration of time.

16. The method of claim 10, which comprises detecting and quantifying the proteins selenoprotein P (SEPP1), alpha-2-Macroglobulin (A2M), angiotensinogen (AGT), complement C1r subcomponent like (C1RL), cluster of differentiation 14 (CD14), ficolin-2 (FCN2), SERPINA1, SERPINF2, and apolipoprotein A1 (APOA1) in the serum sample, measuring a decrease in the concentrations of the proteins over the duration of time, and determining that the subject suffers from upper motor neuron-dominant ALS.

17. The method of claim 10, which comprises detecting and quantifying two or more of the proteins APOA2, APOD, APOL1, C4B, CLEC3B, A2M, AGT, APOA2, C1RL, CD14, FCN2, SERPINA1, SERPINF2, or APOA1 in the serum sample, measuring a decrease in the concentrations of the proteins over the duration of time, and determining that the subject suffers from upper motor neuron-dominant ALS.

18. The method of claim 17, wherein the subject is a human female and the method further comprises detecting and quantifying the APO4 protein in the serum sample and measuring a decrease in the concentrations of APO4 over the duration of time.

19. The method of any one of claims 8-18, wherein the duration of time is about 3 to 5 years.

20. The method of any one of claims 8-19, wherein the treatment comprises riluzole, edaravone, nusinersen, muscle relaxants, botulinum toxin, amitriptyline, glycopyolate, atropine, dextromethorphan, quinidine, anticonvulsants, nonsteroidal anti-inflammatory drugs (NSAIDs), antidepressants, morphine, opiates, physical therapy, occupational therapy, speech therapy, psychotherapy, or any combination of the foregoing.

21. A method of treating a subject suffering from a motor neuron disease, which method comprises:
(a) detecting and quantifying the amount of at least two proteins in a serum sample obtained from a subject suffering from a motor neuron disease, which two or more proteins are selected from apolipoprotein C3 (APOC3), apolipoprotein F (APOF), complement C8 beta chain (C8B), complement C8 gamma chain (C8G), immunoglobulin heavy constant gamma 3 (IGHG3), inter-alpha-trypsin inhibitor heavy chain 3 (ITIH3), quiescin sulfhydryl oxidase 1 (QSOX1), SERPINA10, SERPINA5, von Willebrand factor (VWF), apolipoprotein A2 (APOA2), apolipoprotein A4 (APOA4), apolipoprotein D (APOD), apolipoprotein L1 (APOL1), complement C4-B (C4B), C-type lectin domain family 3 member B (CLEC3B), clusterin (CLU), amyloid P component, serum (APCS), butyrylcholinesterase (BCHE), complement C1R (C1R), complement factor H (CFH), glycoprotein 1 b platelet subunit alpha (GP1BA), protein S (PROS1), SERPINA4, selenoprotein P (SEPP1), alpha-2-Macroglobulin (A2M), angiotensinogen (AGT), complement C1r subcomponent like (C1RL), cluster of differentiation 14 (CD14), ficolin-2 (FCN2), SERPINA1, SERPINF2, apolipoprotein A1 (APOA1), and insulin like growth factor binding protein 3 (IGFBP3),
(b) detecting an increase or decrease of each of the two or more proteins in the sample as compared to the amounts of the same two or more proteins in a control sample, and diagnosing the subject as having a motor neuron disease; and
(c) administering to the subject a treatment for the motor neuron disease.

22. The method of claim 21, wherein the motor neuron disease is amyotrophic lateral sclerosis (ALS), progressive bulbar palsy, pseudobulbar palsy, primary lateral sclerosis (PLS), progressive muscular atrophy, spinal muscular atrophy (SMA), hereditary spastic paraplegia (HSP), or post-polio syndrome (PPS).

23. The method of claim 21 or claim 22, wherein the serum sample is obtained from a subject suffering from amyotrophic lateral sclerosis (ALS).

24. The method of any one of claims 21-23, wherein the treatment comprises riluzole, edaravone, nusinersen, muscle relaxants, botulinum toxin, amitriptyline, glycopyolate, atropine, dextromethorphan, quinidine, anticonvulsants, nonsteroidal anti-inflammatory drugs (NSAIDs), antidepressants, morphine, opiates, physical therapy, occupational therapy, speech therapy, psychotherapy, or any combination of the foregoing.

25. A panel of biomarkers comprising an isolated set of 1000 or fewer cDNA biomarkers, wherein said isolated set includes two or more cDNA biomarkers selected from apolipoprotein C3 (APOC3), apolipoprotein F (APOF), complement C8 beta chain (C8B), complement C8 gamma chain (C8G), immunoglobulin heavy constant gamma 3 (IGHG3), inter-alpha-trypsin inhibitor heavy chain 3 (ITIH3), quiescin sulfhydryl oxidase 1 (QSOX1), SERPINA10, SERPINA5, von Willebrand factor (VWF), apolipoprotein A2 (APOA2), apolipoprotein A4 (APOA4), apolipoprotein D (APOD), apolipoprotein L1 (APOL1), complement C4-B (C4B), C-type lectin domain family 3 member B (CLEC3B), clusterin (CLU), amyloid P component, serum (APCS), butyrylcholinesterase (BCHE), complement C1R (CIR), complement factor H (CFH), glycoprotein 1b platelet subunit alpha (GP1BA), protein S (PROS1), SERPINA4, selenoprotein P (SEPP1), alpha-2-Macroglobulin (A2M), angiotensinogen (AGT), complement C1r subcomponent like (C1RL), cluster of differentiation 14 (CD14), ficolin-2 (FCN2), SERPINA1, SERPINF2, apolipoprotein A1 (APOA1), and insulin like growth factor binding protein 3 (IGFBP3).

26. The panel of biomarkers of claim 25, wherein the cDNA biomarkers span an exon-exon junction.

27. The panel of biomarkers of claim 25, wherein the cDNA biomarkers are full-length cDNAs.
